(19) United States
(12) Patent Application Publication
(10) Pub. No.: US 2025/0064836 A1
(43) Pub. Date: Feb. 27, 2025

(54) COMPOSITIONS AND METHODS FOR TREATING CLOSTRIDIOIDES DIFFICILE INFECTIONS

(71) Applicant: The Children's Medical Center Corporation, Boston, MA (US)

(72) Inventors: Meenakshi Rao, Brookline, MA (US); Min Dong, Weatogue, CT (US)

(73) Assignee: The Children's Medical Center Corporation, Boston, MA (US)

(21) Appl. No.: 18/807,854

(22) Filed: Aug. 16, 2024

Related U.S. Application Data
(60) Provisional application No. 63/520,600, filed on Aug. 18, 2023.

(51) Int. Cl. A61K 31/675; A61K 31/438; A61K 31/444; A61K 31/473; A61K 31/496; A61K 31/517; A61K 31/5377; A61K 31/55; A61K 45/06; A61P 31/04; C07K 16/28

(52) U.S. Cl. CPC A61K 31/675; A61K 31/438; A61K 31/444; A61K 31/473; A61K 31/496; A61K 31/517; A61K 31/5377; A61K 31/55; A61K 45/06; A61P 31/04; C07K 16/28

(57) ABSTRACT

Provided herein are methods of treating a Clostridioides difficile (C. difficile) infection in a subject. In some embodiments, a method of treating a C. difficile infection comprises administering an antagonist of neurogenic inflammation to a subject.

### RELATED APPLICATIONS

This application claims the benefit under 35 U.S.C. § 119 (e) of U.S. provisional application No. 63/520,600, filed Aug. 18, 2023, which is incorporated be reference herein in its entirety.

### FEDERALLY SPONSORED RESEARCH

This invention was made with government support under Grant Number W81XWH-18-1-0639, awarded by the Department of the Army. The Government has certain rights in the invention.

### BACKGROUND

Clostridioides difficile (C. difficile) infection is a leading cause of gastrointestinal infections in developed countries, with an estimated half million cases and ˜29,000 deaths annually in the United States. C. difficile is a spore-forming opportunistic pathogen that can cause colonic infection when the normal gut microbiota is disrupted. CDI-associated diseases present with diarrhea and abdominal pain, and can result in pseudomembranous colitis and toxic megacolon in severe cases. Treatment remains challenging due to frequent recurrent infections, antibiotic resistance, and emerging hypervirulent strains. At tissue levels, CDI is associated with exaggerated inflammatory responses featuring oedema and neutrophil infiltration, leading to colonic epithelial damage and formation of surface plaques filled with neutrophils. Exaggerated immune responses are associated with worse clinical outcomes.

C. difficile causes disease largely through the action of two homologous toxins, toxin A (TcdA) and TcdB. These toxins are large multi-domain proteins that target and enter host cells via receptor-mediated endocytosis, cross the endosomal membrane, and deliver an enzymatic domain into the cytosol of cells. The enzymatic domains of TcdA and TcdB are glucosyltransferases (termed GTD) that modify a key residue on small GTPases and inhibit their activity, leading to disruption of the actin cytoskeleton that eventually results in cell rounding.

Recent studies in mouse models demonstrated that CDI-associated colonic inflammation is toxin-dependent and that toxin-mediated inflammation benefits C. difficile by liberating favorable sources of nutrients and modifying microbiome community structure. However, pathogenic mechanisms that lead to severe colonic inflammation remain unclear.

### SUMMARY

The present disclosure relates to the discovery that exaggerated colonic inflammation caused by Clostridioides difficile toxins such as toxin B (TcdB) damages tissues and promotes Clostridioides difficile infection (CDI), a major cause of healthcare-associated gastrointestinal infections. The inventors identified that antagonists of neurogenic inflammation (e.g., antagonists of a neuropeptide such as substance P or calcitonin gene-related peptide (CGRP)) are effective in treating CDI.

Accordingly, provided herein, in some aspects, is a method of treating a Clostridioides difficile infection in a subject. In some embodiments, a method of treating a C. difficile infection comprises administering a therapeutically effective amount of an antagonist of neurogenic inflammation to the subject. In some embodiments, an antagonist of neurogenic inflammation is an antagonist of a neuropeptide or neuropeptide signaling. In some embodiments, a neuropeptide is substance P or calcitonin gene-related peptide (CGRP).

In some embodiments, an antagonist of substance P is a molecular agent that targets a substance P peptide or a neurokinin-1 receptor (NK1R) protein, optionally wherein the molecular agent is a small molecule or an antibody. In some embodiments, an antagonist of substance P is aprepitant, fosaprepitant, netupitant, palonosetron, rolapitant, fosnetupitant, netupitant, or an aprepitant emulsion. In some embodiments, an antagonist of substance P is a molecular agent that targets a gene encoding a substance Por a neurokinin-1 receptor (NK1R), optionally wherein the molecular agent is an inhibitory nucleic acid.

In some embodiments, an antagonist of CGRP is a molecular agent that targets a CGRP or a CGRP receptor, optionally wherein the molecular agent is a small molecule or an antibody. In some embodiments, an antagonist of CGRP is fremanezumab, olcegepant, rimegepant, ubrogepant, erenumab, epitinezumab, galcanenzumab, atogepant, telcagepant, UB-13 or zavegepant. In some embodiments, an antagonist of CGRP is a molecular agent that targets a gene encoding a CGRP or a CGRP receptor, optionally wherein the molecular agent is an inhibitory nucleic acid. In some embodiments, the CGRP is CGRPα or CGRPβ.

In some embodiments, a subject has a C. difficile infection or is suspected of having a C. difficile infection. In some embodiments, a subject has been diagnosed with a C. difficile infection by fecal testing. In some embodiments, a subject has recurrent C. difficile infection. In some embodiments, a subject is a hospitalized patient. In some embodiments, a subject is at risk of contracting a C. difficile infection. In some embodiments, a subject is an immunocompromised patient or at high risk of colitis, optionally wherein the subject has inflammatory bowel disease, optionally wherein the subject is being treated with a broad-spectrum antibiotic.

In some embodiments, administering a therapeutically effective amount of an antagonist of neurogenic inflammation to the subject results in a decreased period of time to resolution of the infection, relative to a control. In some embodiments, administering a therapeutically effective amount of an antagonist of neurogenic inflammation to the subject results in a decreased incidence of recurrence following resolution of the infection, relative to a control. In some embodiments, a method further comprises administering a standard of care treatment to the subject. A standard of care treatment can be an antibiotic treatment, optionally a vancomycin, fidaxomicin, and/or metronidazole treatment.

### BRIEF DESCRIPTION OF DRAWINGS

FIGS. 1A-1F show SP- and CGRP-mediated neurogenic inflammation in a TcdB cecum injection model and in a CDI mouse model. FIGS. 1A-1B show a time course of histopathological changes after injection of TcdB into the cecums of mice. Representative micrographs of Haemotoxylin and Eosin (H&E) stained tissues are shown in FIG. 1A and histopathology scores are shown in FIG. 1B. Scoring criteria and the sub-categories of histopathology scores are shown in FIGS. 5A-5N; n=5, 6, 9, 5 mice. FIGS. 1C-1D show TcdB was injected into the cecums of wild type (WT) mice or the following KO mice: Tac1 (encoding the precursor for SP), Nk1r (encoding a receptor for SP), Calca (encoding CGRPα), or Calcb (encoding CGRPβ), with representative H&E staining shown in FIG. 1C and histopathology scores shown in FIG. 1D; n=25, 8, 7, 10, 5 mice. FIGS. 1E-1F show CDI experiments were carried out on WT, Tac1 KO, Nk1r KO, and Calcb KO mice. Colon tissues were harvested and analyzed 48 h later, with representative H&E staining shown in FIG. 1E and histopathology scores shown in FIG. IF, n=24, 20, 10, 7 mice. Statistical analyses in FIG. 1B (p<0.001 overall), FIG. 1D (p<0.001 overall), and FIG. 1F were one-way ANOVAs with post-hoc Dunnett's test compared with vehicle-treated mice. P values: n.s., not significant, or exact values shown if <0.05. Centre line indicates mean and error bars reflect the standard error of the mean (SEM). Scale bar: 50 μm. “n” is described from left to right for each graph.

FIGS. 2A-2M show TcdB targets neurons via FZD1/2/7 and pericytes via CSPG4. FIG. 2A shows single cell expression analysis of FZD1, 2 and 7 in human colon tissues (red high, blue low). FIG. 2B shows cultured mouse DRG neurons were exposed to TcdB or TcdB mutants for 1 h. n=10, 7, 7, 7, 3, 3 culture wells across 3 cultures (cach from 5 pooled animals). SP secretion was assessed using ELISA. FzM: TcdB mutant deficient in binding to FZD1/2/7;GtdM: TcdB mutant with mutations in the GTD domain. FIG. 2C shows mouse cecal explants were exposed to TcdB (6 μg, 6 h), TcdB^{FzM}, or saline, and secretion of SP was measured. n=6,6,5 mice. FIG. 2D shows single cell analysis of CSPG4 expression in human colonic stroma. FIGS. 2E-2F show immunostaining of cecum tissues from Cspg4-DsRed mice shows Cspg4⁺ cells (DsRed, cyan) surrounded by CD31+vessels (magenta). FIG. 2E: Whole mount muscularis view, FIG. 2F: cross section. Scale bar: 50 μm. FIG. 2G shows cultured human brain pericytes were exposed to TcdB or TcdB mutants for 5 h and cell rounding (%) was assessed. Cspg4M: TcdB mutant deficient in binding to CSPG4; FzCspg4M: a TcdB double mutant deficient in binding to FZD1/2/7 and CSPG4. n=3 independent cultures. FIG. 2H shows TcdB, but not TcdB-Cspg4M, induced secretion of IL-8 from cultured human pericytes. n=4,7,3,4 from 2 independent cultures. FIGS. 21-2J show TcdB, but not TcdB-Cspg4M, increased CXCL1 in colonic explants after cecal TcdB injections in WT, but not Cspg4 KO mice. In FIG. 21, n=11, 13, 8 mice, and in FIG. 2J, n=5, 8, 4, 4, 3 mice. FIG. 2K shows immunostaining of colon tissues from Cspg4-dsRed mice shows extensive overlap and contacts of neurons (Tubb3, magenta) with Cspg4⁺ cells (cyan). DAPI: yellow. Scale bar: 100 μm. FIG. 21 shows immunostaining of colon tissues from Cspg4-dsRed mice shows appositions of CGRP (yellow) to Cspg4⁺ cells (cyan). Blood vessels were labeled with CD31 (magenta). Scale bar: 10 μm. FIG. 2M shows CGRP induced IL-8 release from cultured human pericytes. n=4 culture wells (representative of 3 replicates). Statistical analyses in FIGS. 2B-2C: one way ANOVA (<0.0001 overall) with post-hoc Dunnett's test compared with vehicle-treated mice, n.s., not significant, p values <0.05 shown. Centre line: mean; error bars: SEM. n is described from left to right.

FIGS. 3A-3J show delivery of TcdB-GTD into peptidergic sensory neurons using the toxogenetic approach is sufficient to induce neurogenic inflammation. FIG. 3A shows mouse footpad injection assays were carried out for the indicated toxins or reagents. Footpad oedema was measured and shown as AThickness. N=4, 3, 8, 4,4,4,4,4 mice. FIG. 3B shows footpad injection assays were carried out on WT, Tac1^{-/-}, C-kit^{W-sh} (deficient in mast cells), and Cspg4^{-/-}mice. N=5, 3, 3, 3 mice. FIG. 3C shows a schematic illustration of the toxogenetic approach. PNS: peripheral nervous system. ENS: enteric nervous system. DRG: dorsal root ganglion. FIG. 3D shows DTR was expressed in Tac1⁺ peptidergic neurons through intravenous injection of AAV.PHP.s.FLEX.DTR: GFP virus into Tac1-Cre mice. Footpad injection assays were carried out. GTD-ciDT: TcdB-GTD fused to a detoxified diphtheria toxin (iDT); iGTD-ciDT: catalytic inactive form of TcdB-GTD fused to ciDT; n=5, 6, 3, 3 mice. FIGS. 3E-3F show intraperitoneal administration of GTD-ciDT induced colonic tissue damage and inflammation in Tac1-Cre mice transduced with AAV.PHP.s.FLEX.DTR: GFP. Representative H&E staining of colonic tissues are shown in FIG. 3E, and histopathology scores are shown in FIG. 3F; n=7, 6, 4 mice. FIGS. 3G-3H show ablating TRPV1⁺ sensory neurons using RTX reduced the levels of SP and histopathology scores in the TcdB cecal injection model; n=7, 10, 10 mice for FIG. 3G and n=10, 10 mice for FIG. 3H. FIGS. 31-3J show systemic administration of GTD-ciDT induced colonic tissue damage and inflammation in Nav1.8^{Cre/+} Rosa-LSL-DTR mice; n=7, 9 mice. Statistical tests in FIG. 3A (p<0.001 overall), FIG. 3B (p<0.001 overall), FIG. 3D (p<0.001), and FIG. 3F (p<0.0001) were one-way ANOVAs. Post-hoc Dunnett's test compared with TcdB treatment (FIG. 3A) or WT treated with TcdB (FIG. 3B). Post-hoc Tukey's tests as indicated in FIG. 3D and FIG. 3F. P values are exact or n.s.; Centre line: mean; error bars: SEM. Scale bars are 50 μm. n is described from left to right.

FIGS. 4A-4K show inhibiting SP or CGRP signaling alleviates inflammation and reduces C. difficile colonization for endemic and hypervirulent epidemic strains. FIGS. 4A-4F show CDI were carried out (630 Δerm strain), and colony forming units (CFUs) of C. difficile in faeces were assessed and plotted in FIG. 4A; n=5, 5 mice. Microbiome diversity was assessed and presented as Fisher's alpha diversity in FIG. 4B; n=18, 16, 5, 10 mice. FIG. 4C shows cecum injection assays were carried out with TcdB on mice pretreated with vehicle, aprepitant, olcegepant, or fremanczumab; n=15, 12, 11, 3. FIGS. 4D-4E show CDI were carried out (630 Δerm) on mice pretreated with vehicle, aprepitant, olcegepant, or fremanezumab; n=13, 7, 8, 8 mice in FIG. 4D; n=3 (d1) and n-6 (d2) in FIG. 4E. FIGS. 4F-4G show TcdB2 induces oedema in WT and Cspg4^{-/-} in the footpad injection assay; n=3, 4, 4 mice. Aprepitant and olcegepant can prevent oedema induced by TcdB2. TcdB2: heat inactivated TcdB2, n=5, 5, 4 mice. FIGS. 4H-4I show CDI with the strain M7404 (TcdA-/TcdB2) of mice pre-treated with vehicle, aprepitant, or olcegepant. Survival is plotted in FIG. 4H, n=31, 28, 21. Histological scores are plotted in FIG. 4I, n=3, 9, 7 mice. FIGS. 4J-4K show CDI with the hypervirulent strain R20291 of mice pre-treated with the indicated inhibitor (Combination F+A: fremanezumab plus aprepitant; Combination O+A: olcegepant plus aprepitant). Histopathological scores are shown in FIG. 4J, n=21, 10, 10, 12, 10, 5 mice; CFUs are shown in FIG. 4K, n=21, 9, 10, 12, 10, 5 mice. Statistical analyses for FIGS. 4A-4B and FIG. 4E: two-way ANOVA, variables are time and genotype/drug treatment, post-hoc Sidak's test with comparisons by day. For FIGS. 4C-4D, FIGS. 4F-4G, and FIGS. 4I-4K: one-way ANOVA (<0.0001 overall) with post-hoc Dunnett's test compared with vehicle (FIGS. 4C-4D, FIG. 4G, and FIGS. 4I-4K). For FIG. 4H, drugs were compared by Fisher's test to vehicle controls. Centre line: mean; error bars: SEM; n.s., not significant; p values are exact. n is described from left to right.

FIGS. 5A-5N show histopathology score analysis and time course of TcdB-induced inflammation in a mouse cecum injection model. FIG. 5A shows a list of criteria for assessing histopathology scores of colonic tissues. FIGS. 5B-5E show H&E micrographs illustrating oedema (FIG. 5B, black line indicates oedema extent); epithelial disruption (FIG. 5C); haemorrhage or congestion (FIG. 5D); immune cell infiltration (FIG. 5E). FIG. 5F shows representative histopathology of human CDI colon tissues compared to a normal control, illustrating neutrophilia, oedematous inflammation, and pseudomembranes. FIGS. 5G-5J show the subcategories for the histopathology scoring in FIG. 1B: oedema (FIG. 5G), immune cell infiltration (FIG. 5H), epithelial disruption (FIG. 5I), and haemorrhage or congestion (FIG. 5J). n=5, 6, 9, 5 mice. FIGS. 5K-5L show SP (FIG. 5K) and CGRP (FIG. 5L) levels in colonic explants from the mouse cecal TcdB injection model at the indicated time (min) following TcdB injection. Vehicle was at 120 min. n=6, 7, 7, 7, 7 mice. FIGS. 5M-5L show the levels of SP (FIG. 5M) and CGRP (FIG. 5N) in colonic explants from CDI mouse models infected with cither C. difficile 630 or a toxin-null strain (630 A-B-) at 23 h (n=5), 37 h (n=5), and 48 h (n=3), normalized to mice treated with antibiotics but gavaged with saline (time point 0, n=5). FIGS. 5G-5J assessed by one-way ANOVA with post hoc Dunnett's test. FIGS. 5K-5L assessed by Kruskall-Wallis test with post-hoc Dunn's test. Centre line, mean; error bars reflect the standard error of the mean (SEM); n.s., not significant, p values are exact. Scale bar is 50 μm in FIGS. 5B-5C and 20 μm in FIGS. 5D-5E. n is described from left to right.

FIGS. 6A-6V show histological scoring for cecum injections of TcdB in the indicated mouse models as described in FIGS. 1C-1D. FIGS. 6A-6E show histopathology total scores and subscores of TcdB injected Tac1 KO mice, n=7, 8 mice. FIGS. 6F-6J show histopathology total scores and subscores of TcdB injected Nklr KO mice, n=7, 7 mice. FIGS. 6K-60 show histopathology total scores and subscores of TcdB injected Calca KO mice, n=8, 10 mice. FIGS. 6P-6T show histopathology total scores and subscores of TcdB injected Calcb KO mice, n=3, 5 mice. FIG. 6U show representative histopathology of Tac1 KO mice injected with TcdB compared to Tac1 KO mice injected with TcdB and SP in the cecal TcdB injection model (120 min incubation). FIG. 6V show histopathology scores of experiments described in FIG. 6U, n=3, 4 mice. Assessed by Student's T-test, two tailed, relative to WT control mice. Centre line: mean; error bars: SEM; n.s., not significant, p values are exact. n is described from left to right.

FIGS. 7A-7O show histological scoring for CDI in the indicated KO mouse models as described in FIGS. 1E-1F. FIGS. 7A-7O show histopathology total scores and subscores of CDI in Tac1 KO mice, n=11, 20 mice. FIGS. 7F-7J show histopathology total scores and subscores of CDI in Nk1r KO mice, n=7, 10 mice. FIGS. 7K-7O show histopathology total scores and subscores of CDI in Calcb KO mice, n=6, 9, 7 mice. FIGS. 7A-7J, assessed by Student's T-test, two tailed, relative to WT control mice. FIGS. 7K-70, assessed by one-way ANOVA with post hoc Dunnett's test. Centre line: mean; error bars: SEM; n.s., not significant, p values are exact. n is described from left to right.

FIGS. 8A-8E show FZD1/2/7 are expressed in DRG and enteric neurons. FIG. 8A shows single cell RNA-seq analysis of FZD1, 2, and 7 expression in different cell types within colonic tissues in mice. Size of dot indicates proportion expressing whereas the colour indicates expression level (red high, blue low). Putative excitatory motor neuron (PEMN), putative inhibitory motor neuron (PIMN), putative sensory neuron (PSN), putative sudomotor and vasodilator neurons (PSVN), putative interneuron (PIN). FIG. 8B shows FZD1, 2, and 7 expression in DRG neurons that innervate colonic tissues are displayed as violin plots. FIG. 8C shows in situ hybridization of spinal cord sections shows enriched expression of FZD1/2/7 in dorsal root ganglia areas. Data are from Allen Brain Atlas. FIG. 8D shows phase-contrast images show that DRG neurons exposed to TcdB (1 pM, 60 min) exhibit no morphological changes. Scale bar is 50 μm. n=3 independent replicates. FIG. 8E shows RNAscope in situ hybridization showing localization of Pdgfrb expression (magenta) within Cspg4⁺ cells (cyan, Cspg4-dsRed mice) in colonic tissues. Panels in bottom row show regions in yellow boxed area at higher magnification. Scale bar: 50 μm and lower panel is 20 μm.

FIGS. 9A-9L show Csog4⁺ pericytes wrap around blood vessels and form close contact with CGRP-containing afferent neurons. FIG. 9A shows colon tissues from Cspg4-DsRed mice were harvested and subjected to whole mount immunofluorescence analysis, using an antibody against CD31 (a marker for blood vessel endothelial cells; magenta). DsRed (cyan) signals were detected directly. DsRed-positive cells surround CD31-positive capillaries in colonic crypts. Maximum intensity projection of the whole stack is presented in FIG. 2E. Scale bar is 50 μm. FIG. 9B shows experiments were carried out as in FIG. 9A, except that lymphatic endothelial cells were labeled with an antibody against Lyve-1 (magenta) and DsRed signals were detected using an antibody against DsRed. DsRed-positive cells are clearly separate from lymphatic endothelial cells. Scale bar is 50 μm. FIGS. 9C-9F show experiments were carried out as described in FIG. 9A. Enteric glial cells were visualized with GFP fluorescence in examining proteolipid protein 1 (PLP1)-GFP/-Cspg4-dsRed dual reporter mice (FIG. 9C). Fibroblasts were labeled using an antibody against platelet-derived growth factor receptor alpha (PGDFRA, magenta, FIG. 9D). Myofibroblasts were labeled using an antibody against smooth muscle actin (SMA, magenta, FIG. 9E). Mast cells and interstitial cells of Cajal were labeled using an antibody against cKit (magenta, FIG. 9F). Scale bar: 10 μm. FIG. 9G shows TcdB induced cell-rounding of cultured human brain vascular pericytes. Scale bar: 25 μm. FIG. 9H shows the indicated TcdB mutants were injected into mouse cars via intradermal injection. Pericytes surrounding car arterioles were labeled and visualized through DsRed. TcdB and TcdB-FzM induced morphological changes of Csog4⁺ pericytes surrounding car arterioles, whereas TcdB-Cspg4M showed no effect. Scale bar, 50 μm. n=3 mice/group. FIG. 91 shows experiments were carried out as described in FIG. 9H, except that WT mice were utilized and pericytes were detected by immunostaining using an antibody against smooth muscle actin (labeling pericytes). TcdB-FzM disrupted pericytes around ear arterioles, whereas TcdB-Cspg4M has no effect. Scale bar: 50 μm. n=3 mice/group. FIG. 9J shows experiments were carried out as in FIG. 9A, except that neuronal processes were labeled using an antibody against Tubb3 (magenta, a marker for neuronal processes), showing that neuronal processes are extending alongside DsRed-positive pericytes. Scale bar: 10 μm. FIG. 9K show experiments were carried out as in FIG. 9A, except that an antibody against CGRP was added to detect CGRP-positive nerve terminals. 3-dimensional reconstruction of images showed that DsRed⁺ pericytes (cyan) surround the vasculature (CD31 endothelial marker; magenta) and contact CGRP-expressing nerve terminals (CGRP; yellow). Scale bar: 10 μm. FIG. 9L shows immunoblot analysis of cell lysates showed expression of CGRP receptors (CALCRL: calcitonin receptor like receptor; and RAMP1: receptor activity modifying protein 1) in primary cultured human brain vascular pericytes. Two human cell lines, HeLa and U87, were analyzed in parallel as controls, which do not express detectable levels of CGRP receptors. Total protein staining with Coomassie blue was used as a control for protein loading. N=2 replicates.

FIGS. 10A-10H show AAV-DTR mediates selective expression of DTR in ENS and DRG neurons in Tac1-Cre mice. FIG. 10A shows AAV-DTR was administered to Tac1-Cre mice via tail vein injection. Colon tissues were harvested 3-weeks later and subjected to whole mount immunofluorescence analysis. DRG were harvested and their cryosections were analyzed. HuC/D (magenta) marks neuron soma in colon tissues. An antibody against β3-tubulin (TUBB3, magenta) was utilized to label DRG neurons. DTR-GFP was labeled using an antibody against GFP (cyan). Scale for DRG: 100 μm; for myenteric plexus: 50 μm. Images were collected using a confocal microscope and maximal intensity projection is presented. GFP is directly fused to the C-terminus of DTR, thus cyan color represents DTR expression. n=4 mice. FIG. 10B shows experiments were carried out as described. 5-HT (magenta) labels enterochromaffin cells in colonic tissues, showing no co-expression of DTR-GFP (cyan). Scale bars represent 100 μm (50 μm for right panel, which shows boxed region at higher magnification). FIGS. 10C-10D show experiments were carried out as described in FIG. 3E. Colonic tissues (FIG. 10C) and DRG (FIG. 10D) were isolated and subjected to analysis as described in FIG. 10A. WT mice not exposed to AAV-DTR were analyzed in parallel as a negative control (right panels). HuC/D labels myenteric plexus neurons within the proximal colon and TUBB3 labels DRG neurons of a wildtype mouse injected with AAV-DTR. No GFP positive cell bodies or fibers were detected in WT mice. Scale bar is 50 μm. FIGS. 10E-10H show sub-scoring of histopathology, related to FIG. 3F, n=7, 6, 4 mice. One-way ANOVAs were applied with post hoc Tukey's test as indicated. Centre line: mean; error bars: SEM; n.s., not significant, p values are exact. n is described from left to right.

FIGS. 11A-11Q show extrinsic afferent neuron-dependent secretion of SP is necessary and sufficient for TcdB mediated pathology. FIGS. 11A-11D show histopathology subscoring of experiments following TcdB administration to RTX treated mice. Related to FIG. 3H, n=10, 10 mice. FIGS. 11E-11J show histopathology of RTX treated/vehicle mice treated with TcdB alone or TcdB and SP for 120 min, demonstrating that SP can restore the effects of TcdB in RTX-treated mice. Representative histopathology in FIG. 11E, and histopathology scores in FIGS. 11F-11J, n=4, 4, 6, and 6 mice. The scale bar is 50 μm. FIG. 11K shows CGRP secretion from RTX treated mice is reduced in the cecal TcdB injection model and is restored by co-administration of SP with TcdB. n=2, 4, 6, 4, and 6 mice. FIGS. 11L-11M show Nav1.8^{Cre/+} Rosa-LSL-Tdtomato mice have no evident Tomato expression (magenta) in the ENS (myenteric neurons marked with HuC/D, cyan) with only sparse Tomato⁺ nerve fibers from extrinsic neurons labeled in the colon (FIG. 11L). In contrast, there are many Tomato⁺ neurons in DRG (FIG. 11N). Scale bar: 50 μm. FIGS. 11N-11Q show histopathology subscores of Nav1.8^{Cre/+} Rosa-LSL-DTR mice treated with GTD-iDT related to FIGS. 31-3J; n=7, 9 mice. For FIGS. 11A-11D and FIGS. 11N-11Q, significance was assessed by student's two-tailed t-test. For FIGS. 11F-11K, significance was assessed by one-way ANOVA with post hoc Dunnett's test. Centre line: mean; error bars: SEM. p values are exact. n is described from left to right.

FIGS. 12A-12L show inhibiting SP or CGRP signaling reduces severity of C. difficile colitis. FIG. 12A shows CDI models were carried out on WT versus Tac1 KO mice, using either C. difficile 630 Δerm or a toxin-null mutant strain (A-/B-). C. difficile CFUs from faeces were analyzed at 2 days post infection. The toxin-null strain showed reduced colonization on WT mice compared with the standard 630 Δerm, suggesting that the presence of toxins facilitates C. difficile colonization. In contrast, 630 Δerm showed similar levels of CFUs as the toxin-null strain in Tac1 KO mice, suggesting that the toxin's contribution to C. difficile colonization relies on intact SP signaling. n=7, 6, 4, 3 mice. FIG. 12B shows CDI models were carried out on WT versus Calcb KO mice (using C. difficile 630 Δerm). C. difficile CFUs from faeces showed reduced colonization in Calcb KO mice compared with WT mice, n=4, 5 mice. FIG. 12C shows representative H&E images for the experiments described in FIG. 4C. Scale bar is 50 μm. FIGS. 12D-12G show sub-categories of histopathological scoring of FIG. 4C n=15, 12, 11, 3. FIGS. 12H-12L show histopathology scoring of mice administered vehicle or aprepitant at the indicated times (0, 30 or 60 min following cecum injection and wound closure); n=7, 6, 6, 7 mice. Significance was assessed for FIG. 12A—Two-Way ANOVA with post hoc Sidak's test and by T-test for FIG. 12B. One Way ANOVA with post hoc Dunnett's test was used for FIGS. 12D-12L. Centre line: mean; error bars: SEM. p values are exact. n is described from left to right.

FIGS. 13A-13K show inhibiting SP or CGRP reduces histopathological scores in cecum injection assays and in CDI models. FIG. 13A shows representative H&E images for the experiment described in FIG. 4D. Scale bar is 50 μm. FIGS. 13B-13E show sub-categories of histopathological scoring of FIG. 4D, n=13,7, 8, 8 mice. FIG. 13F C. difficile 630 Δerm fitness (measured by OD600) was not affected by treatment with aprepitant or olcegepant (20 μM) in vitro. Representative of 2 independent experiments (3 wells/group). FIGs. 13G-13H show CDI experiments (with C. difficile 630) were carried out on WT and Tac1 KO mice (co-housed from weaning), followed by treatment with vehicle or aprepitant. C. difficile colonization (FIG. 13G) and histopathological scores (FIG. 13H) were shown, n=7, 7, 6, 7 mice. FIGS. 13I-13J show CDI experiments (with C. difficile 630) were carried out on WT and Calcb KO mice (littermates, co-housed from birth), followed by treatment with vehicle or olcegepant. C. difficile colonization (FIG. 131) and histopathological scores (FIG. 13J) were shown. n=4, 4, 5, 7 mice. FIG. 13K shows TcdA induced oedema in footpad injection assays in mice, which is prevented by pre-treatment with aprepitant. Boiled TcdA solution did not induce any oedema. n=7,4, 3 mice. Significance was assessed by one-way ANOVA with post hoc Dunnett's test. Centre line: mean; error bars: SEM. n.s., not significant, p values are exact. n is described from left to right.

FIGS. 14A-14J show inhibiting SP or CGRP reduces histopathological scores and colonization of hypervirulent strains that express TedB2. FIGS. 14A shows representative H&E images for the experiments described in FIG. 4I. Scale bar is 50 μm. FIGS. 14B-14E show sub-categories of histopathological scoring of FIG. 4I, N=3, 9, 7 mice. FIG. 14F shows representative H&E images for the experiment described in FIG. 4J. Scale bar is 50 μm. FIGS. 14G-14J show sub-categories of histopathological scoring of FIG. 4J, n=21, 10, 10, 12, 10, 5 mice. Significance was assessed by one-way ANOVA with post hoc Dunnett's test. Centre line: mean; error bars: SEM; n.s., not significant; p values are exact. n is described from left to right.

### DETAILED DESCRIPTION

Neurogenic inflammation is a well-established phenomenon in which sensory neurons release pro-inflammatory neuropeptides such as substance P (SP) or calcitonin gene-related peptide (CGRP), causing rapid vasodilation and increased vascular permeability that results in extravasation of plasma and immune cells into the tissue. SP is well known to act on the endothelium to induce plasma extravasation, and the role of CGRP in vasodilation is also well established. Symptoms consistent with neurogenic inflammation in CDI have been observed, including neutrophilic infiltration, extreme oedema, and increased colonic vascular permeability. Provided herein are methods for treating CDI in subject through modulation of neurogenic inflammation.

The present disclosure provides methods for treating a Clostridioides difficile infection in a subject in need thereof, the method comprising administering to the subject a therapeutically effective amount of any of antagonists of neurogenic inflammation or a pharmaceutical compositions comprising the same, as provided herein. In some embodiments, administering a therapeutically effective amount of an antagonist of neurogenic inflammation to a subject results in a decreased period of time to resolution of a C. difficile infection (e.g., relative to a control). For example, administering a therapeutically effective amount of an antagonist of neurogenic inflammation to a subject can result in a decrease of at least 1, 2, 3, 4, 5, 6, or 7 days to resolution of a C. difficile infection (e.g., relative to a control). In some embodiments, administering a therapeutically effective amount of an antagonist of neurogenic inflammation to a subject can result in a decrease of at least 1, 2, 3, or 4 weeks to resolution of a C. difficile infection (e.g., relative to a control). In some embodiments, administering a therapeutically effective amount of an antagonist of neurogenic inflammation to a subject (or a population of subjects) results in a decreased incidence of recurrence following resolution of the infection (e.g., relative to a control). For example, administering a therapeutically effective amount of an antagonist of neurogenic inflammation to a population of subjects can result in a decreased incidence of recurrence of a C. difficile infection in at least 1, 2, 3, 4, 5, 6, or 7 subjects in the population (e.g., relative to a control).

An antagonist of neurogenic inflammation is, in some embodiments, an antagonist of a neuropeptide or neuropeptide signaling. In some embodiments, an antagonist of a neuropeptide is a compound or molecule (or molecular agent) that functions to downregulate and/or inhibit neuropeptide production (e.g., neuropeptide expression) in cells (e.g., cells of a subject). In some embodiments, an antagonist of neuropeptide signaling is a compound or molecule (or molecular agent) that functions to downregulate and/or inhibit neuropeptide signaling in cells (e.g., cells of a subject).

In some embodiments, delivery of an antagonist of neurogenic inflammation to a subject results in a reduction in the level of neurogenic inflammation in the subject of at least 10%, 20%, 30%, 40%, 50%, 60%, 70%, 80%, 90%, 100%, 200% or 500% relative to a control (e.g., the subject before delivery of the antagonist or a subject who has been received the antagonist). In some embodiments, delivery of an antagonist of a neuropeptide (e.g., Substance Por CGRP) to a subject results in a reduction in the level of expression of the neuropeptide in the subject of at least 10%, 20%, 30%, 40%, 50%, 60%, 70%, 80%, 90%, 100%, 200% or 500% relative to a control (e.g., the subject before delivery of the antagonist or a subject who has been received the antagonist). In some embodiments, delivery of an antagonist of neuropeptide signaling (e.g., Substance Por CGRP signaling) to a subject results in a reduction in the level of neuropeptide signaling in the subject of at least 10%, 20%, 30%, 40%, 50%, 60%, 70%, 80%, 90%, 100%, 200% or 500% relative to a control (e.g., the subject before delivery of the antagonist or a subject who has been received the antagonist).

A neuropeptide is a small peptide or polypeptide chain of amino acids that are synthesized and released by neurons. Neuropeptides typically bind to G protein-coupled receptors (GPCRs) and function to modulate neural activity and other tissues like the gut, muscles, and heart. In some embodiments, a neuropeptide is bradykinin, calcitonin gene-related peptide (CGRP), cholecystokinin, enkephalin, galanin, neurotensin, neuropeptide Y, somatostatin, substance P (SP), or vasopressin. In some embodiments, a CGRP is a CGRPα isoform or a CGRPβ isoform.

Substance P is, in some embodiments, a neuropeptide composed of a chain of 11 amino acids and belongs to the tachykinin family of neuropeptides. In some embodiments, an antagonist of substance P is a molecular agent that targets a substance P peptide or a neurokinin-1 receptor (NK1R) protein. An antagonist of substance P can be a small molecule or an antibody.

In some embodiments, an antagonist of substance P is aprepitant, fosaprepitant, netupitant, palonosetron, rolapitant, fosnetupitant, netupitant, or an aprepitant emulsion.

In some embodiments, an antagonist of substance P is as provided in Table 1.

TABLE 1
Chemical structure of Substance P antagonists

| Small molecule | |
|---|---|
| name | Chemical Structure |
| aprepitant | 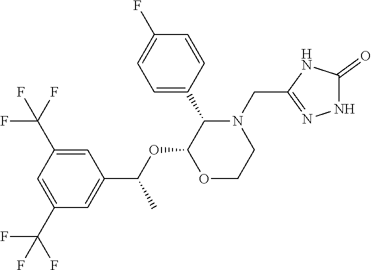 [SMILES: C[C@@H](O[C@H]1OCCN(Cc2n[nH]c(=O)[nH]2)[C@H]1c1ccc(F)cc1)c1cc(C(F)(F)F)cc(C(F)(F)F)c1] |
| fosaprepitant | 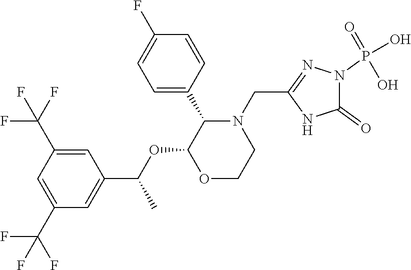 [SMILES: C[C@@H](O[C@H]1OCCN(Cc2nn(P(=O)(O)O)c(=O)[nH]2)[C@H]1c1ccc(F)cc1)c1cc(C(F)(F)F)cc(C(F)(F)F)c1] |
| netupitant | 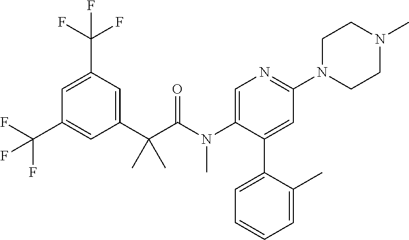 [SMILES: Cc1ccccc1-c1cc(N2CCN(C)CC2)ncc1N(C)C(=O)C(C)(C)c1cc(C(F)(F)F)cc(C(F)(F)F)c1] |
| palonosetron | 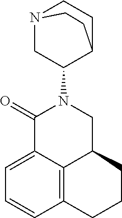 [SMILES: O=C1c2cccc3c2[C@H](CCC3)CN1[C@@H]1CN2CCC1CC2] |
| rolapitant | 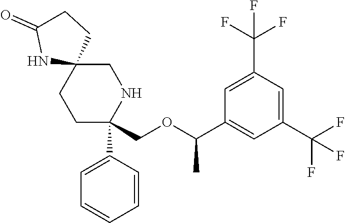 [SMILES: C[C@@H](OC[C@@]1(c2ccccc2)CC[C@]2(CCC(=O)N2)CN1)c1cc(C(F)(F)F)cc(C(F)(F)F)c1] |
| fosnetupitant | 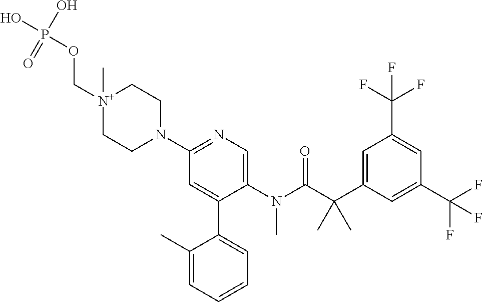 [SMILES: Cc1ccccc1-c1cc(N2CC[N+](C)(COP(=O)(O)O)CC2)ncc1N(C)C(=O)C(C)(C)c1cc(C(F)(F)F)cc(C(F)(F)F)c1] |

In some embodiments, aprepitant and/or fosaprepitant are as described in Aapro et al., Oncologist. 2015 April;20 (4): 450-8. In some embodiments, netupitant and/or aprepitant emulsions are described in Karthaus et al., Expert Rev Clin Pharmacol. 2019 July; 12 (7): 661-680. In some embodiments, palonosetron is as described in Celio et al., Core Evid. 2015 Aug. 21;10:75-87. In some embodiments, rolapitant is as described in Rapoport, Rev Recent Clin Trials. 2017;12 (3): 193-201. In some embodiments, fosnetupitant is as described in Abe et al., Adv Ther. 2023 May;40 (5): 1913-1925.

In some embodiments, an antagonist of substance P is a molecular agent that targets a gene encoding a substance P or a neurokinin-1 receptor (NK1R). A molecular agent can be an inhibitory nucleic acid that targets a gene encoding a substance Por a neurokinin-1 receptor (NK1R).

In some embodiments, an inhibitory nucleic acid that targets a gene encoding substance P binds to (e.g., is complementary to) the gene encoding substance P via a region of complementarity. In some embodiments, an inhibitory nucleic acid that targets a gene encoding NK1R binds to (e.g., is complementary to) the gene encoding NK1R via a region of complementarity. In some embodiments, an inhibitory nucleic acid that targets a gene encoding NK1R binds to (e.g., is complementary to) a gene as set forth in NCBI RefSeqGene NG_029522.2; NCBI RefSeqGene NM_001058.4; or NCBI RefSeqGene NM_015727.3. For example, binding of an inhibitory nucleic acid to a gene can trigger RNAi pathway-mediated degradation of the gene (in the case of dsRNA, siRNA, shRNA, etc.), or can block the translational machinery (e.g., antisense oligonucleotides). In some embodiments, an inhibitory nucleic acid has a region of complementarity that is complementary with at least 8 nucleotides of an mRNA encoded by a gene encoding substance P or NK1R. Inhibitory nucleic acids can be single-stranded or double-stranded. In some embodiments, inhibitory nucleic acids are DNA or RNA. In some embodiments, an inhibitory nucleic acid is selected from the group consisting of: antisense oligonucleotide, siRNA, shRNA and miRNA. In some embodiments, an inhibitory nucleic acid is a modified nucleic acid.

Calcitonin gene-related peptide (CGRP) is neuropeptide that exists in two natural isoforms—CGRP alpha (CGRPα), and CGRP beta (CGRPβ). CGRPα is a 37-amino acid neuropeptide that is formed by alternative splicing of the calcitonin/CGRP gene located on chromosome 11. CGRPβ differs from CGRPα by three amino acids and is encoded in a different gene than CGRPα. Accordingly, in some embodiments, a CGRP is a CGRPα isoform or a CGRPβ isoform. In some embodiments, an antagonist of CGRP is a molecular agent that targets a CGRP or a CGRP receptor. An antagonist of CGRP can be a small molecule or an antibody.

In some embodiments, an antagonist of CGRP is fremanezumab, olcegepant, rimegepant, ubrogepant, erenumab, epitinezumab, galcanenzumab, atogepant, telcagepant, UB-13 or zavegepant. In some embodiments, fremanezumab is as described in Urits et al., Pain Ther. 2020 June;9(1):195-215.

In some embodiments, an antagonist of CGRP is as provided in Table 2.

TABLE 2
Chemical structure of CGRP antagonists

| Small molecule | |
|---|---|
| name | Chemical Structure |
| olcegepant | 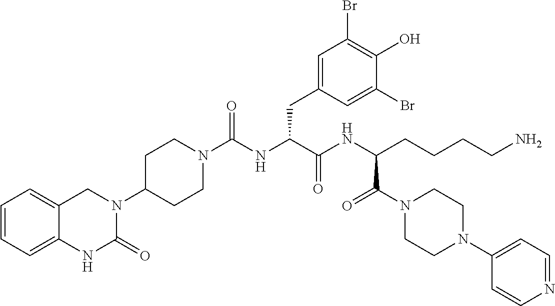 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1] |
| telcagepant | 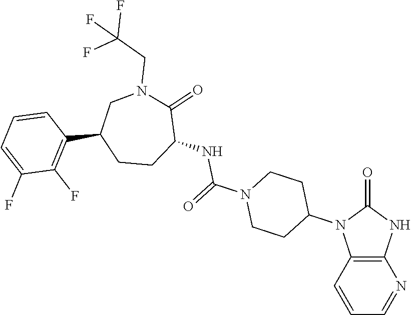 [SMILES: O=C(N[C@@H]1CC[C@@H](c2cccc(F)c2F)CN(CC(F)(F)F)C1=O)N1CCC(n2c(=O)[nH]c3ncccc32)CC1] |
| rimegepant | 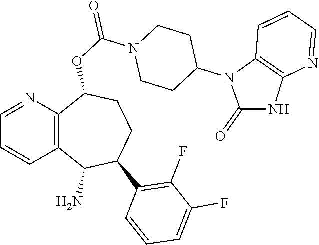 [SMILES: N[C@@H]1c2cccnc2[C@H](OC(=O)N2CCC(n3c(=O)[nH]c4ncccc43)CC2)CC[C@H]1c1cccc(F)c1F] |
| ubrogepant | 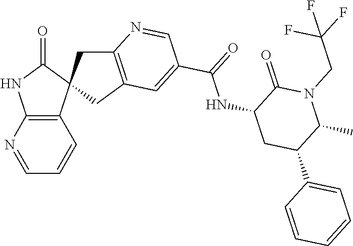 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F] |
| atogepant | 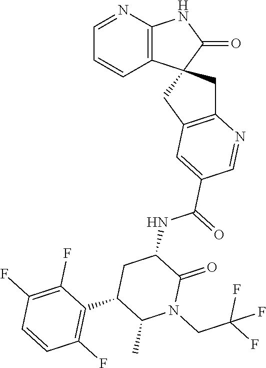 [SMILES: C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F] |
| zavegepant | 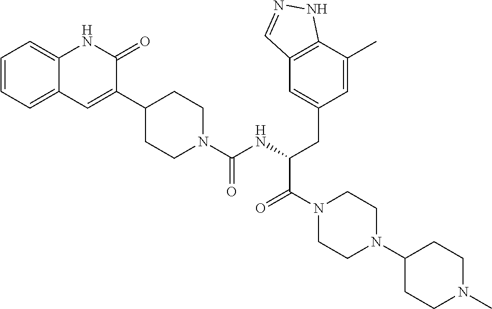 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12] |

In some embodiments, olcegepant and telcagepant are described in Yao et al.,. Neural Regen Res. 2013 Apr. 5;8(10):938-47. In some embodiments, rimegepant is as described in Gao et al., Front Pharmacol. 2020 Jan. 24:10:1577. In some embodiments, ubrogepant is as described in Scott, Drugs. 2020 Feb. 5:80:323-328. In some embodiments, erenumab is as described in Lattanzi et al., Drugs. 2019:79;417-431. In some embodiments, epitinezumab is described in Datta et al., Cureus. 2021 Sep. 16;13(9):e18032. In some embodiments, galcanenzumab is as described in Gklinos et al., Therapeutic Advances in Neurological Disorders. 2020;13. In some embodiments, atogepant is as described in Boinpally et al., Clin. Transl. Sci. 2024 Jan. 10;17(1):e13707. In some embodiments, zavegepant is as described in Khan et al., Cureus. 2023 Jul. 17;15(7):e41991.

In some embodiments, an antagonist of CGRP is a molecular agent that targets a gene encoding a CGRP or a CGRP receptor. A molecular agent can be an inhibitory nucleic acid. In some embodiments, an inhibitory nucleic acid that targets a gene encoding CGRP or a CGRP receptor binds to (e.g., is complementary to) the gene encoding CGRP or a CGRP receptor via a region of complementarity. In some embodiments, an inhibitory nucleic acid that targets a gene encoding CGRP or a CGRP receptor binds to (e.g., is complementary to) the gene encoding CGRP or a CGRP receptor via a region of complementarity. In some embodiments, an inhibitory nucleic acid that targets a gene encoding CGRP binds to (e.g., is complementary to) a gene as set forth in NCBI RefSeqGene NG_015960.1; NCBI NM_001033952.3; NCBI NM_001033953.3; NCBI NM_001378949.1; NCBI NM_001378950.1; NCBI NM_001741.3; or NCBI NM_000728.4. In some embodiments, an inhibitory nucleic acid that targets a gene encoding a CGRP receptor binds to (e.g., is complementary to) a gene as set forth in NCBI NM_001040647.2; NCBI NM_001040648.2; NCBI NM_001142414.1; or NCBI NM_014478.5. For example, binding of an inhibitory nucleic acid to a gene can trigger RNAi pathway-mediated degradation of the gene (in the case of dsRNA, siRNA, shRNA, etc.), or can block the translational machinery (e.g., antisense oligonucleotides). In some embodiments, an inhibitory nucleic acid has a region of complementarity that is complementary with at least 8 nucleotides of an mRNA encoded by a gene encoding substance P or NK1R. Inhibitory nucleic acids can be single-stranded or double-stranded. In some embodiments, inhibitory nucleic acids are DNA or RNA. In some embodiments, an inhibitory nucleic acid is selected from the group consisting of: antisense oligonucleotide, siRNA, shRNA and miRNA. In some embodiments, an inhibitory nucleic acid is a modified nucleic acid.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. In some embodiments, a subject has a Clostridioides difficile infection or is suspected of having a Clostridioides difficile infection.

In some embodiments, a subject has a C. difficile infection or is suspected of having a C. difficile infection. In some embodiments, a subject has been diagnosed with a C. difficile infection by fecal testing. In some embodiments, a subject has recurrent C. difficile infection (e.g., has had more than two, e.g., 3, 4, 5, 6, or more C. difficile infections). In some embodiments, a subject is a hospitalized patient. In some embodiments, a subject is at risk of contracting a C. difficile infection. In some embodiments, a subject is an immunocompromised patient or at high risk of colitis. In some embodiments, a subject has inflammatory bowel disease. In some embodiments, a subject is being treated with a broad-spectrum antibiotic.

A therapeutically effective amount of antagonist of neurogenic inflammation can be administered to a subject in need of treatment. A “therapeutically effective amount” of a composition described herein is an amount sufficient to provide a therapeutic benefit in the treatment of a condition (e.g., a Clostridioides difficile infection) or to delay or minimize one or more symptoms associated with the condition. A therapeutically effective amount of a composition means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a Clostridioides difficile infection.

In certain embodiments, a pharmaceutical composition described herein is provided in an effective amount to a subject. In certain embodiments, the effective amount is a therapeutically effective amount. In certain embodiments, the effective amount is a prophylactically effective amount. In certain embodiments, the effective amount is an amount effective for treating a C. difficile infection in a subject in need thereof. In certain embodiments, the effective amount is an amount effective for preventing the development of a C. difficile infection in a subject in need thereof. In certain embodiments, the effective amount is an amount effective for reducing the risk of developing a C. difficile infection in a subject in need thereof.

The terms “treatment,” “treat,” and “treating” refer to reversing, alleviating, delaying the onset of, or inhibiting the progress of a disease, disorder, or infection described herein, such as a Clostridioides difficile infection. In some embodiments, treatment may be administered after one or more signs or symptoms of the disease, disorder, or infection have developed or have been observed. In other embodiments, treatment may be administered in the absence of signs or symptoms of the disease, disorder, or infection. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

Pharmaceutical compositions described herein can be prepared by any method known in the art of pharmaceutics. In general, such preparatory methods include bringing the components of the composition described herein (i.e., the “active ingredients”) into association with a carrier or excipient, and/or one or more other accessory ingredients, and then, if necessary and/or desirable, shaping, and/or packaging the product into a desired single-or multi-dose unit. Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

The compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical, mucosal, nasal, bucal, sublingual; by intratracheal instillation, bronchial instillation, and/or inhalation; and/or as an oral spray, nasal spray, and/or aerosol.

The pharmaceutical compositions containing a compound can be administered by any suitable route for administering medications. A variety of administration routes are available. The particular mode selected will depend, of course, upon the particular agent or agents selected, the particular condition being treated, and the dosage required for therapeutic efficacy. The methods of this disclosure, generally speaking, may be practiced using any mode of administration that is medically acceptable, meaning any mode that produces therapeutic effect without causing clinically unacceptable adverse effects. Various modes of administration are discussed herein. For use in therapy, an effective amount of the antagonists and/or other therapeutic agent can be administered to a subject by any mode that delivers the agent to the desired surface, e.g., mucosal, systemic.

Administering the pharmaceutical composition of the present disclosure may be accomplished by any means known to the skilled artisan. Routes of administration include but are not limited to oral, parenteral, intravenous, intramuscular, intraperitoneal, intranasal, sublingual, intratracheal, inhalation, subcutaneous, ocular, vaginal, and rectal. Systemic routes include oral and parenteral. Several types of devices are regularly used for administration by inhalation. These types of devices include metered dose inhalers (MDI), breath-actuated MDI, dry powder inhaler (DPI), spacer/holding chambers in combination with MDI, and nebulizers.

For oral administration, the compounds can be formulated readily by combining the active compound(s) with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

A composition, as described herein, can be administered in combination with one or more additional pharmaceutical agents (e.g., therapeutically and/or prophylactically active agents). The compositions can be administered in combination with additional pharmaceutical agents that improve their activity (e.g., activity (e.g., potency and/or efficacy) in treating a disease or infection in a subject in need thereof, in preventing a disease or infection in a subject in need thereof, and/or in reducing the risk of developing a disease or infection in a subject in need thereof. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The composition can be administered concurrently with, prior to, or subsequent to one or more additional pharmaceutical agents, which may be useful as, e.g., combination therapies. In some embodiments, an additional pharmaceutical agent is a standard of care treatment. In some embodiments, a standard of care treatment is an antibiotic treatment. An antibiotic treatment can be a vancomycin, fidaxomicin, and/or metronidazole treatment. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the composition described herein in a single dose or composition or administered separately in different doses or compositions. The particular combination to employ in a regimen will take into account compatibility of the composition described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Also encompassed by the disclosure are kits (e.g., pharmaceutical packs). The kits provided may comprise a pharmaceutical composition described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition described herein. In some embodiments, the pharmaceutical composition described herein provided in the first container and the second container are combined to form one unit dosage form. Thus, in one aspect, provided are kits including a first container comprising a pharmaceutical composition described herein. In certain embodiments, the kits are useful for treating a disease, disorder, or infection (e.g., a C. difficile infection) in a subject in need thereof. In certain embodiments, the kits are useful for preventing a disease, disorder, or infection (e.g., a C. difficile infection) in a subject in need thereof. In certain embodiments, the kits are useful for reducing the risk of developing a disease, disorder, or infection (e.g., a C. difficile infection) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease, disorder, or infection (e.g., a C. difficile infection) in a subject in need thereof. In certain embodiments, the kits and instructions provide for preventing a disease, disorder, or infection (e.g., a C. difficile infection) in a subject in need thereof. In certain embodiments, the kits and instructions provide for reducing the risk of developing a disease, disorder, or infection (e.g., a C. difficile infection) in a subject in need thereof. A kit described herein may include one or more additional pharmaceutical agents described herein as a separate composition.

In some embodiments, an inhibitory nucleic acid can be delivered to the cells via an expression vector engineered to express the inhibitor nucleic acid. An expression vector is one into which a desired sequence may be inserted, e.g., by restriction and ligation, such that it is operably joined to regulatory sequences and may be expressed as an RNA transcript. An expression vector typically contains an insert that is a coding sequence for a protein or for a inhibitory nucleic acid such as an shRNA, a miRNA, or an miRNA. Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays or fluorescent proteins, etc.

### EXAMPLES

### Example 1. C. difficile Intoxicates Neurons and Pericytes to Drive Neurogenic Inflammation

### TcdB Induces Neurogenic Inflammation

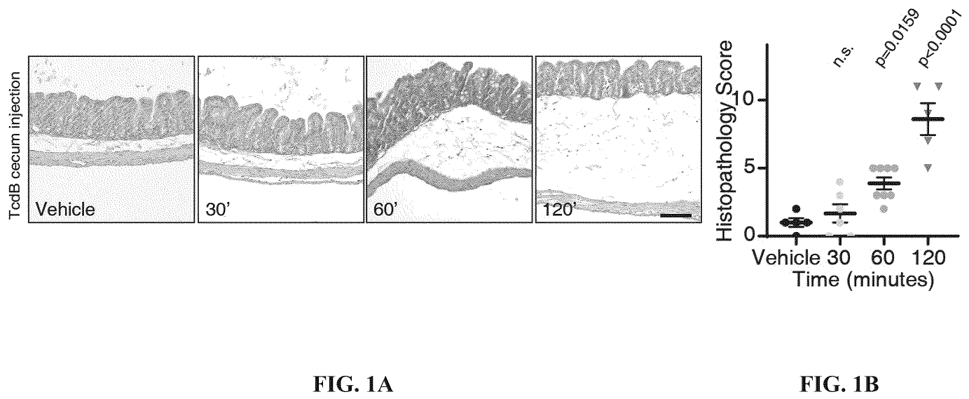

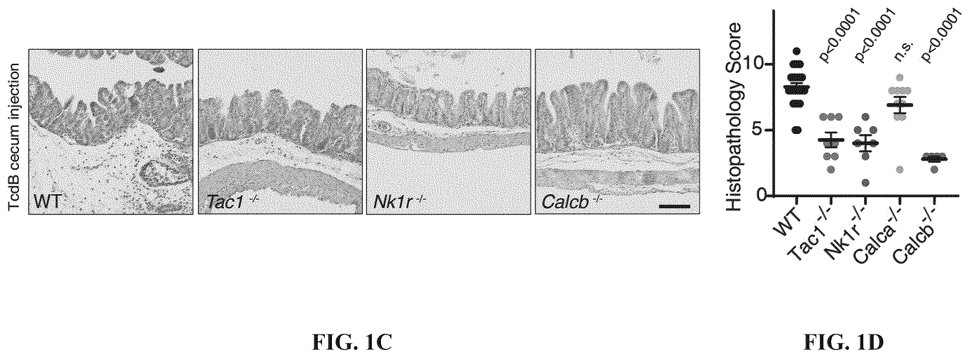

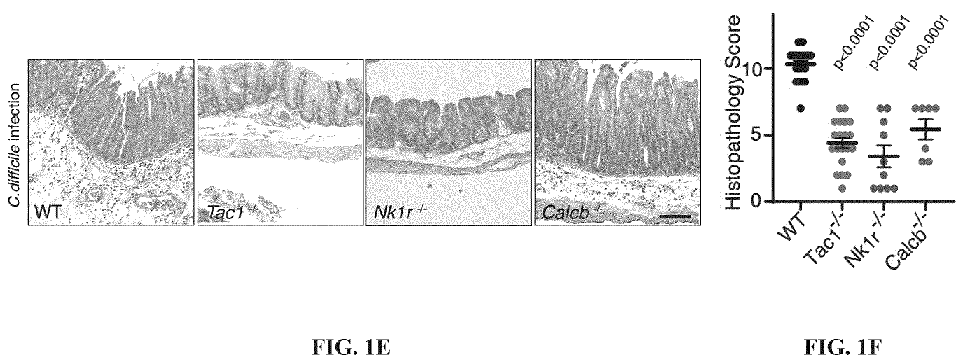

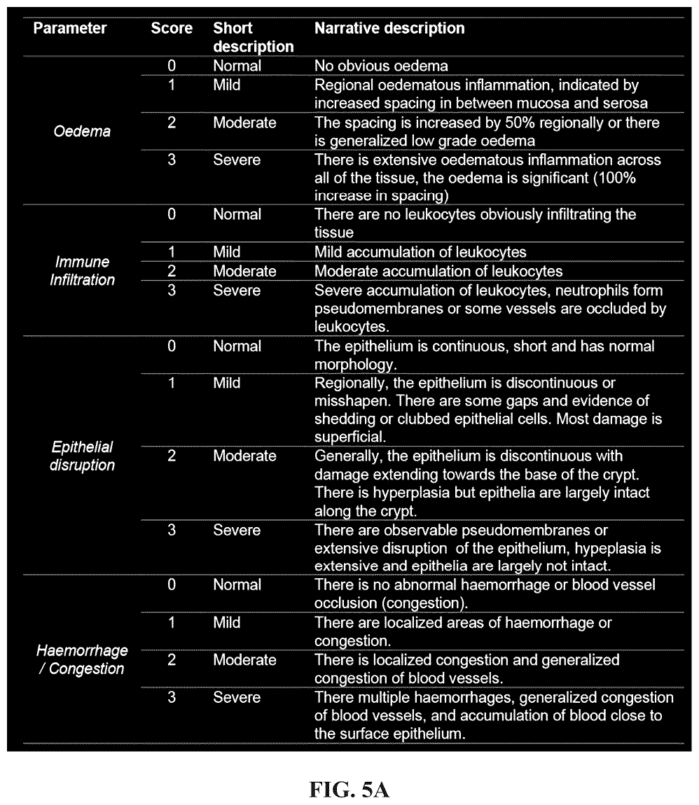

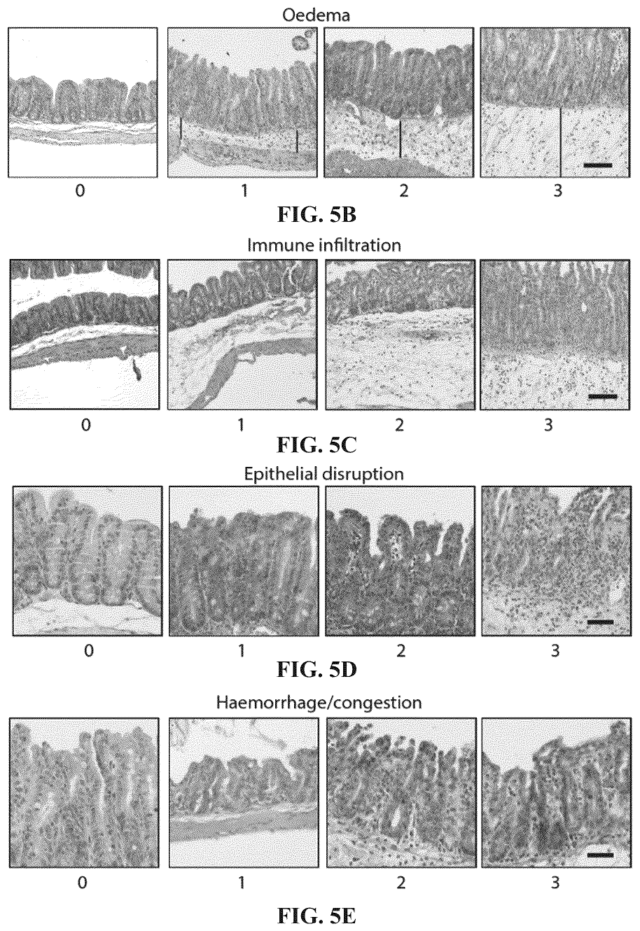

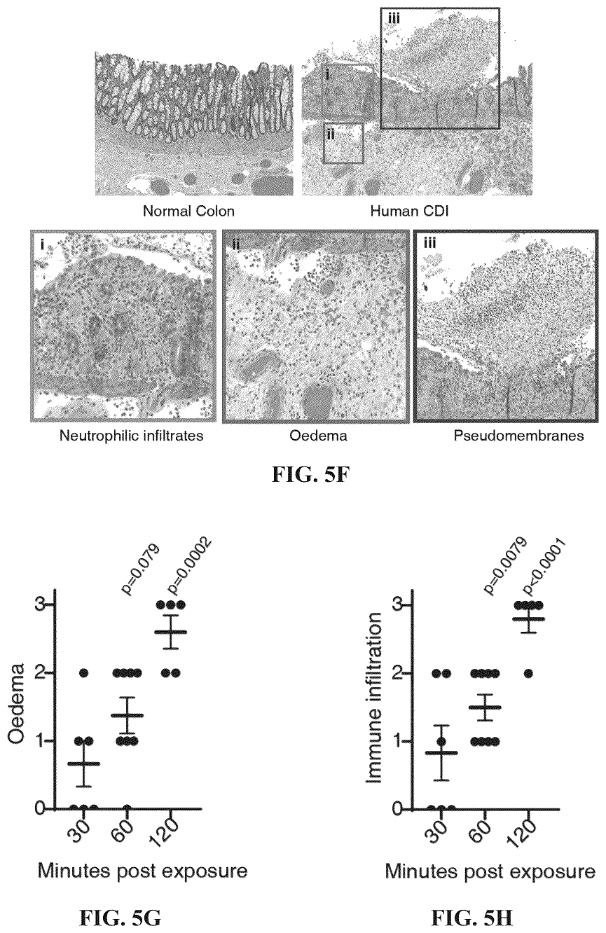

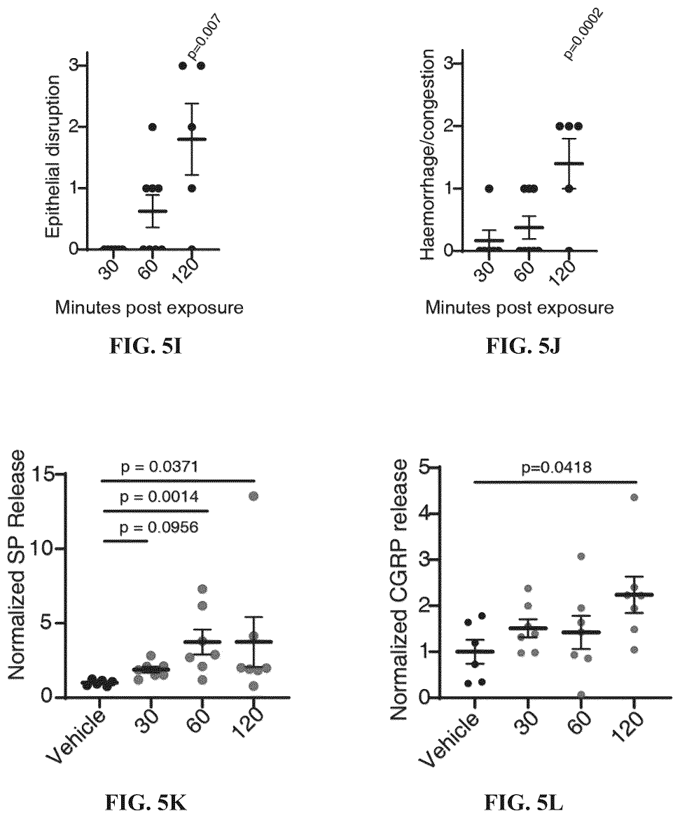

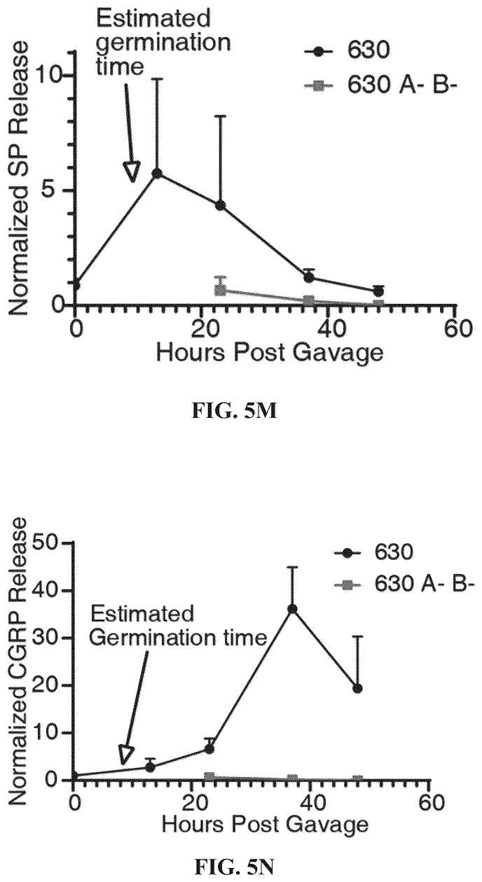

To assess if TcdB induces rapid tissue changes consistent with neurogenic inflammation, TcdB was injected directly into the cecal lumens of wildtype (WT) mice and then isolated tissue for histologic analysis (FIG. 1A). Histopathology was scored based on four criteria: oedema, immune cell (neutrophil) infiltration, epithelium disruption, and tissue hemorrhage (FIGS. 5A-5E), representative of pathologic changes observed in human CDI (FIG. 5F). TcdB induced oedema and neutrophil infiltration within 1 hour (h) (FIGS. 1A-1B, FIGS. 5G-5J), reminiscent of classic neurogenic flare responses in the skin.

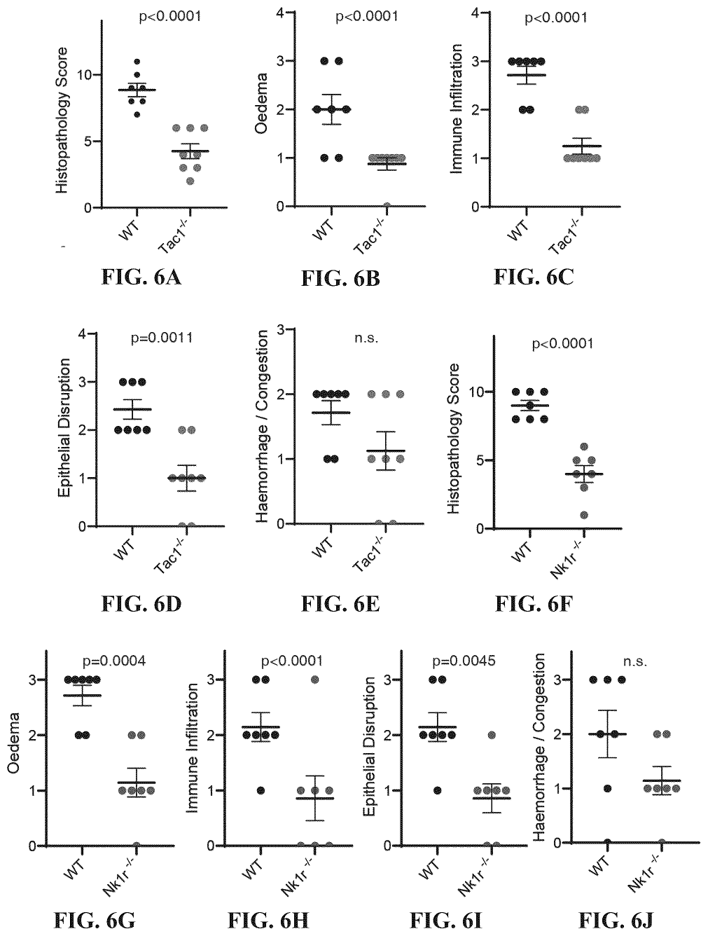

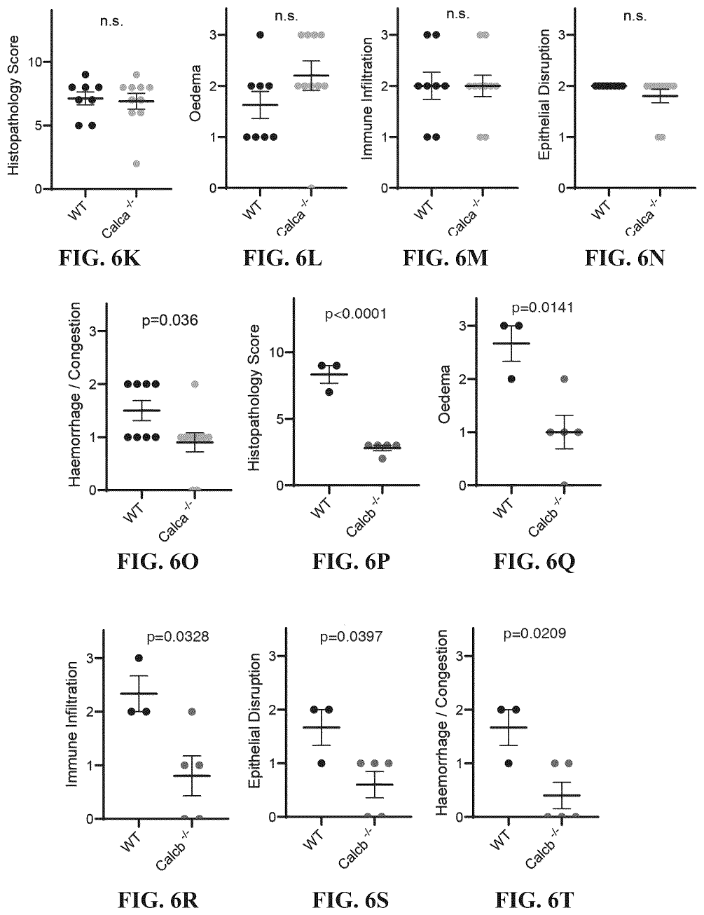

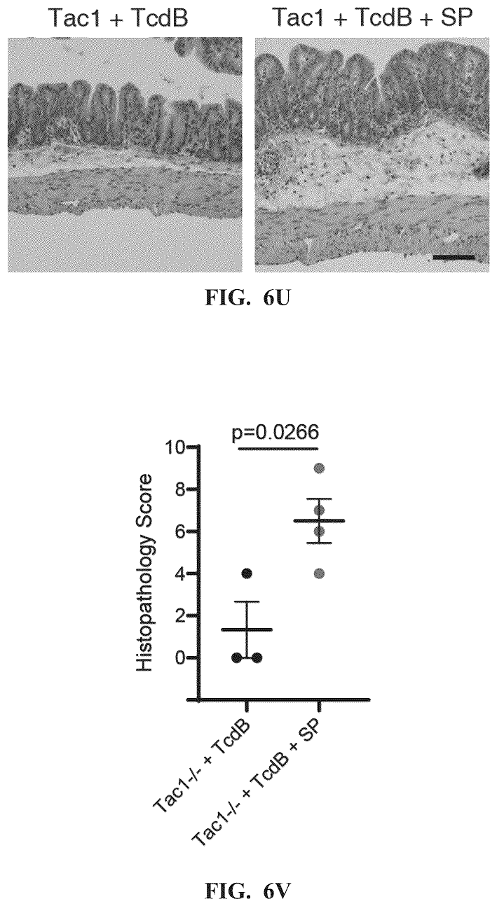

SP and CGRP are key drivers of vasodilation and neutrophil infiltration in the skin and other tissues. The levels of both are increased in cecal tissues after exposure to TcdB, with the rapid timescale correlating with histopathological changes (FIGS. 5K-5L). Next, knockout (KO) mice lacking SP or CGRP signaling were examined. Tac1 (encodes the prepropeptide for SP and neurokinin A) and Nk1r (encodes neurokinin-1 receptor, the high-affinity receptor for SP) KO mice had markedly lower histopathology scores after TcdB injection than WT controls (FIGS. 1C-1D, FIGS. 6A-6J). CGRP has two closely related forms encoded by distinct genes: Calca (CGRPα) and Calcb (CGRPβ). While Calca-null mice had similar histopathology as WT controls, Calcb-null mice showed reduced histopathological scores after TcdB administration (FIGS. 1C-1D, FIGS. 6K-6T). On the other hand, administering SP together with TcdB into the cecal lumen restored the susceptibility of Tac1 mice to TcdB (FIGS. 6U-6V). These data suggest that SP and CGRPβ are critical to the rapid inflammatory response induced by TcdB.

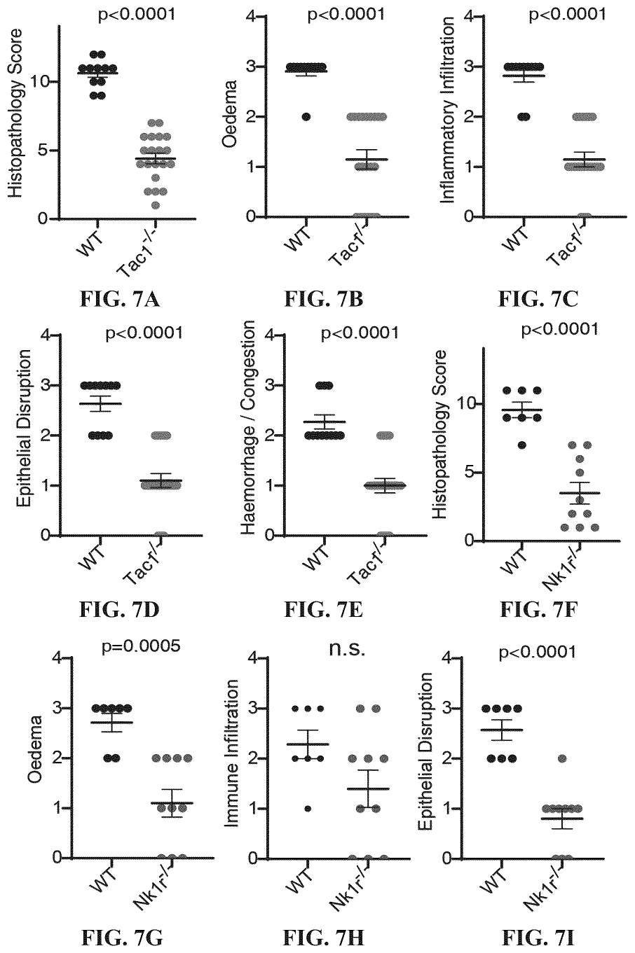

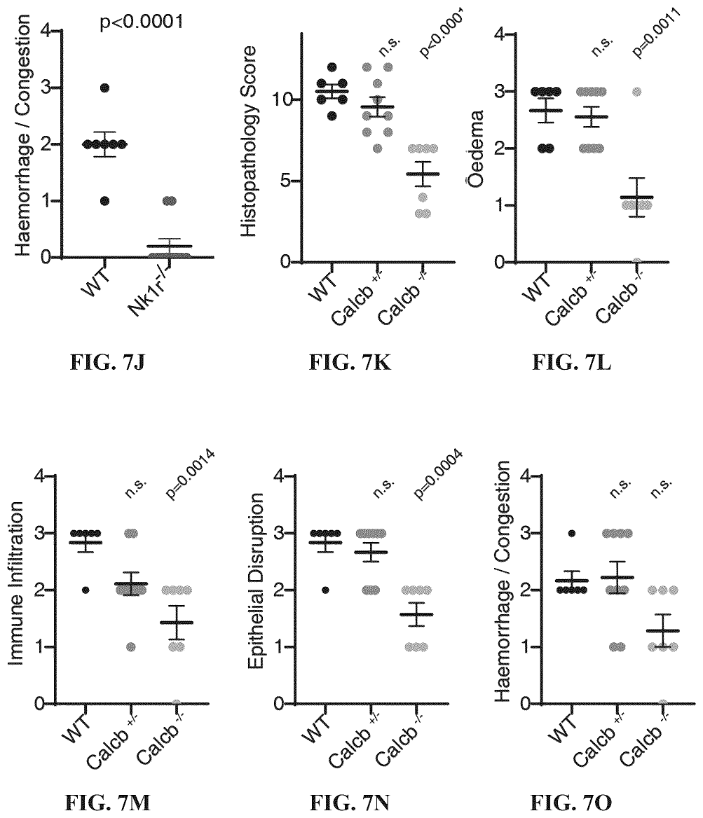

To validate the role of SP and CGRPβ in CDI, an antibiotic treatment mouse model of CDI was utilized. Mice pre-treated with antibiotics were infected with the spores of a widely used C. difficile strain (630 Δerm) that produces TcdA and TcdB. Severe pathology including diarrhea, oedema, neutrophil infiltration, and epithelial damage developed in WT mice within 48 h (FIGS. 1E-IF). Consistently, SP and CGRP levels in colonic tissues rose during the timeframe of infection, but not in mice infected with a toxin-deficient strain (FIGS. 5M-5N). In contrast to WT mice, Tac1, Nk1r, and Calcb KO mice infected with C. difficile 630 Δerm showed lower histopathological scores (FIGS. 1E-IF, FIGS. 7A-7O).

### TcdB Targets Sensory Neurons via FZD1/2/7

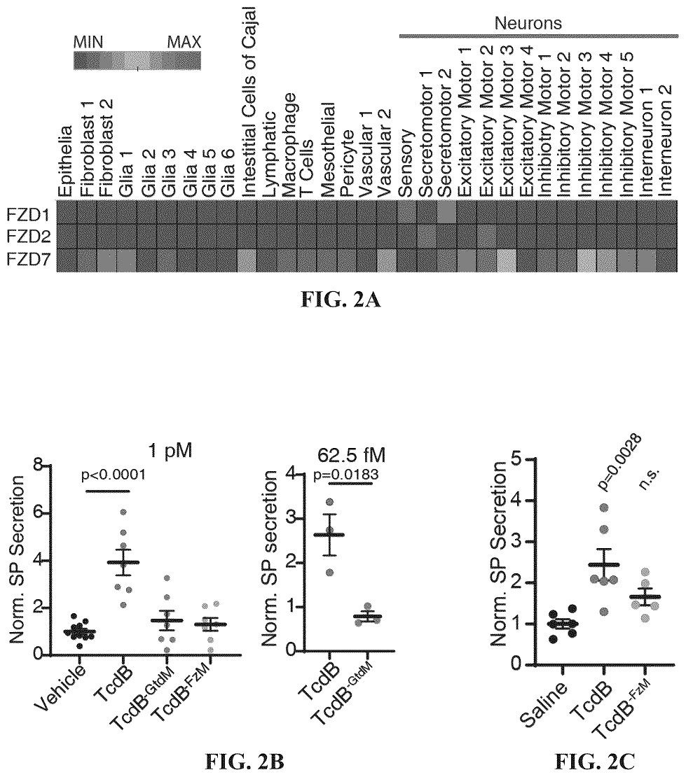

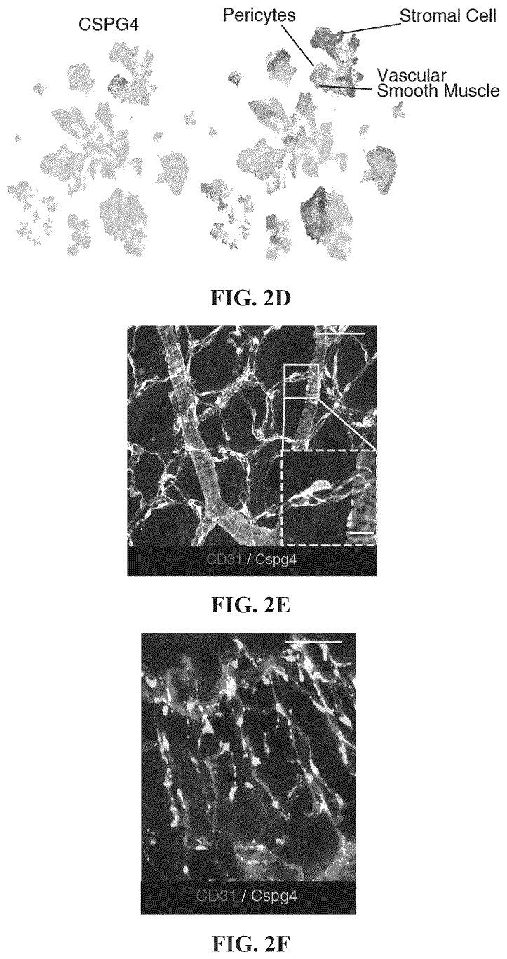

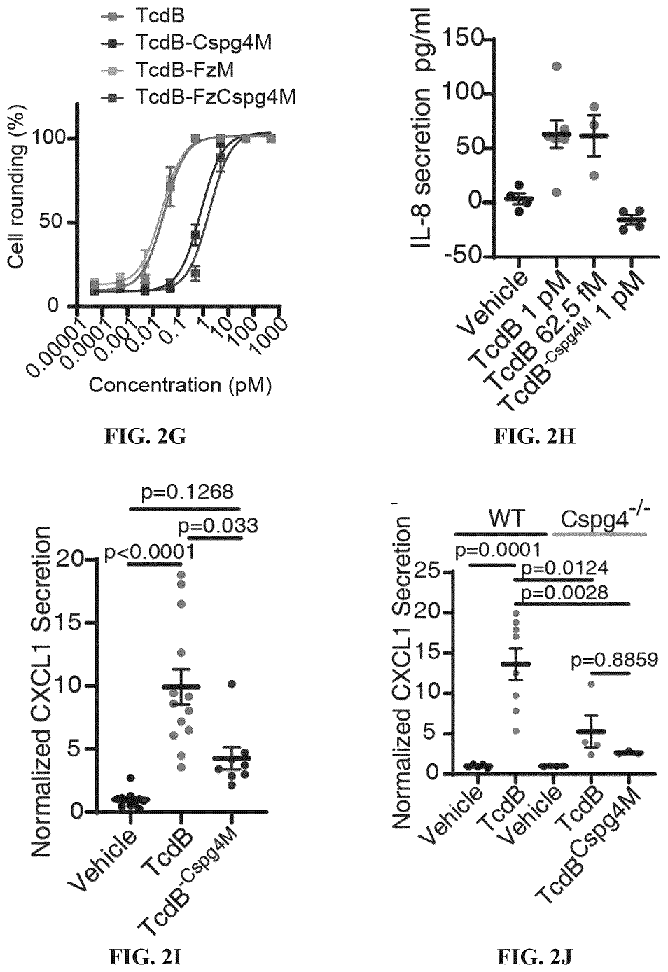

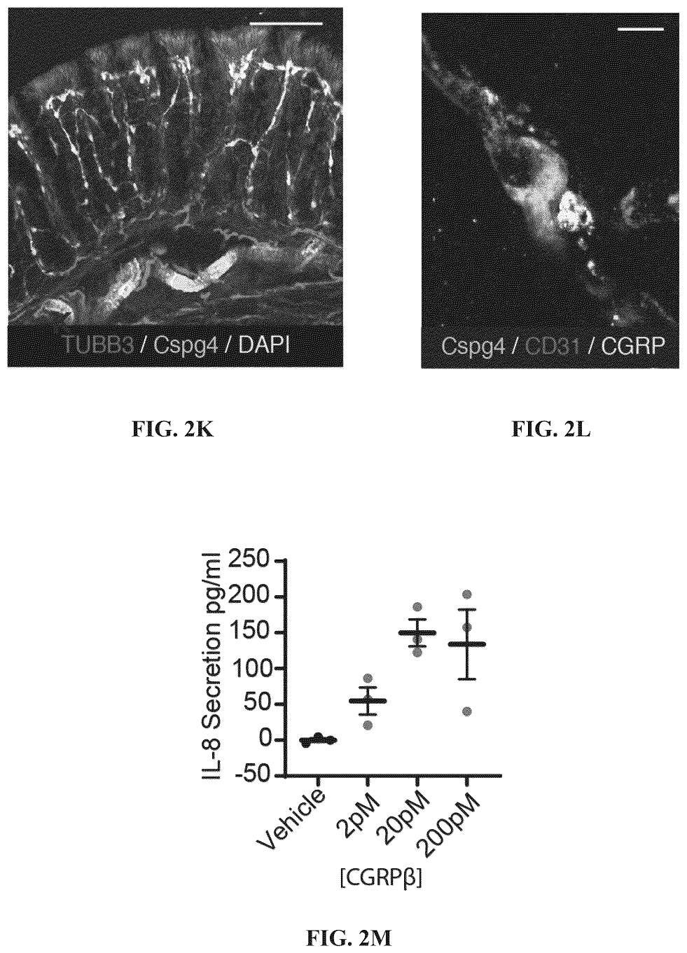

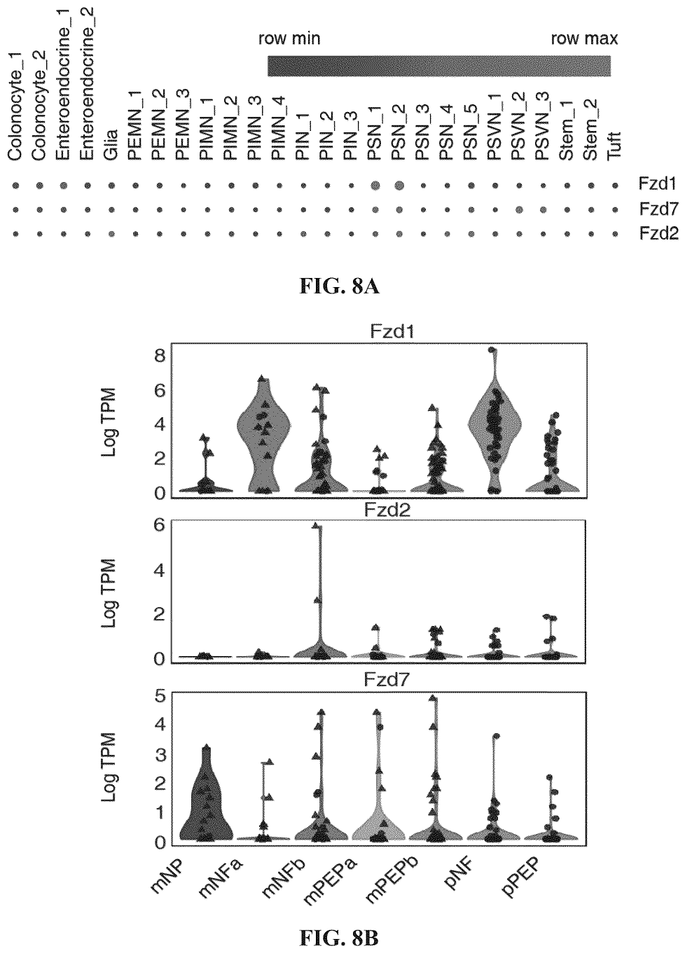

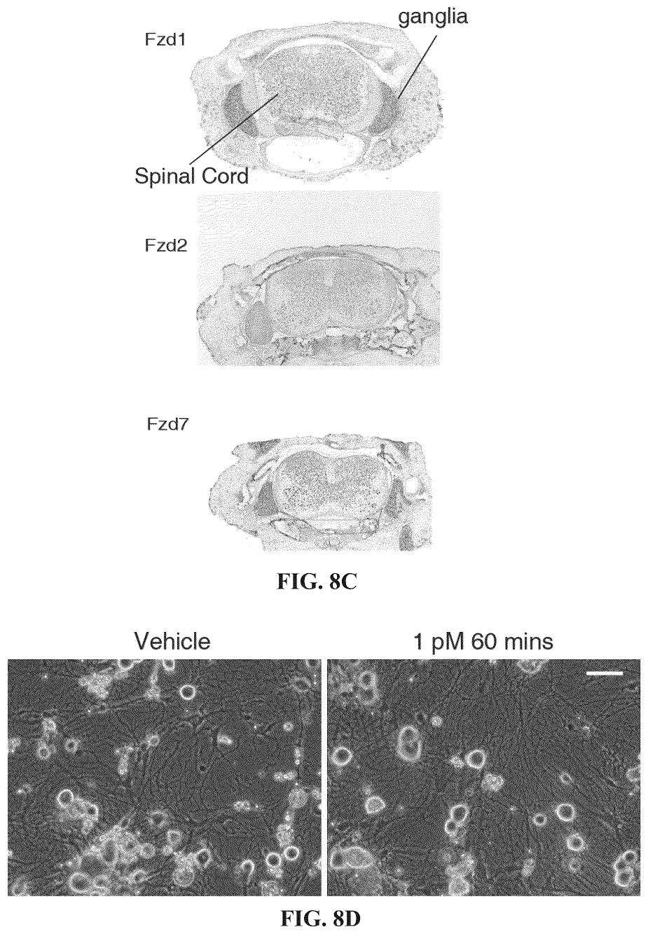

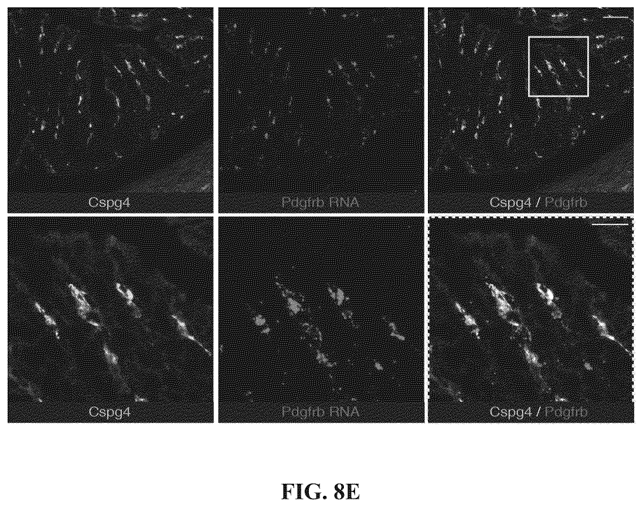

The cecum and colon are innervated by intrinsic neurons of the enteric nervous system (ENS) and projections from extrinsic neurons located in dorsal root ganglia (DRG). FZD1/2/7 and CSPG4 are well-established receptors for TcdB. Analysis of published single cell RNA sequencing (scRNA-seq) data indicated that FZD1/2/7 transcripts are expressed in ENS neurons in mouse and human colon, at levels higher than in many other intestinal cell types including epithelial cells (FIG. 2A, FIG. 8A). FZD1/2/7 have been previously reported in DRG neurons and play a role in sensory neuron sensitization. Single cell RNA sequencing of colon-innervating DRG neurons in mice also showed expression of FZD1/2/7 (FIG. 8B). Moreover, in situ hybridization confirmed that FZD1/2/7 are expressed in the DRG (Allen Brain Atlas; FIG. 8C).

Consistent with expression of FZD1/2/7, it was found that cultured mouse DRG neurons are highly sensitive to TcdB and exposure to TcdB led to SP secretion before visible damage to neurites (FIG. 2B, FIG. 8D). A mutant TcdB with mutations that disrupt glucosylation of small GTPases (TcdB-GtdM) did not induce SP release (FIG. 2B). A TcdB mutant deficient in binding to FZD1/2/7 (TcdB-FzM) was previously generated and validated. This mutant toxin did not induce SP release from DRG neurons (FIG. 2B). The effect of TcdB was further confirmed in vivo: TcdB treatment increased SP release within cecal tissue explants following a mouse cecum injection assay while TcdB-FzM showed reduced potency (FIG. 2C). These findings demonstrate that TcdB targets peripheral sensory neurons via FZD1/2/7 and that intoxication induces neuropeptide secretion.

### TcdB Targets Pericytes via CSPG4

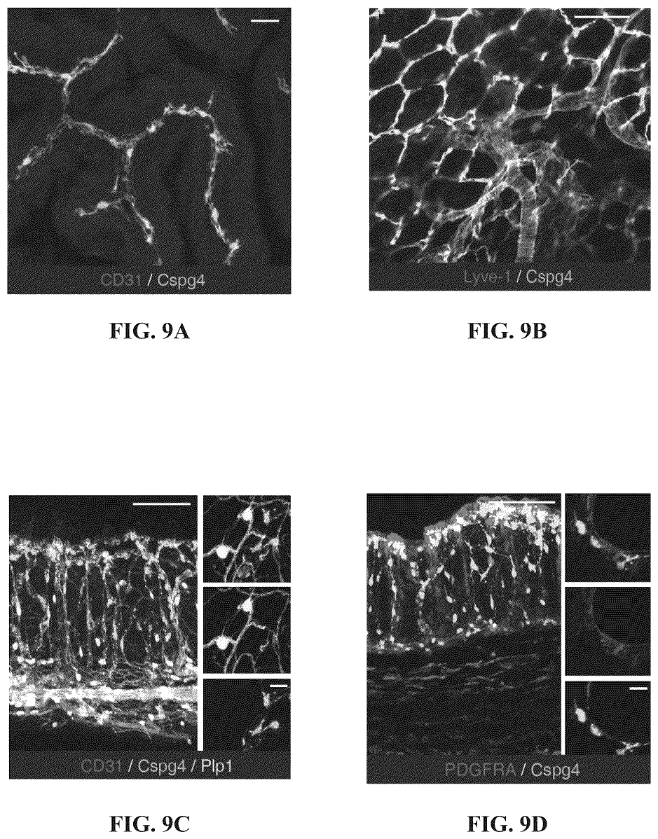

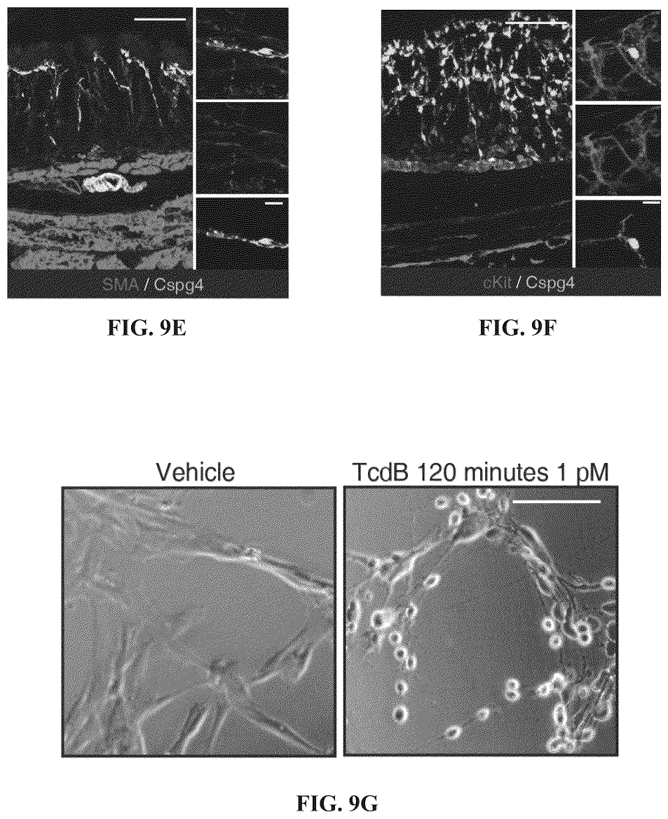

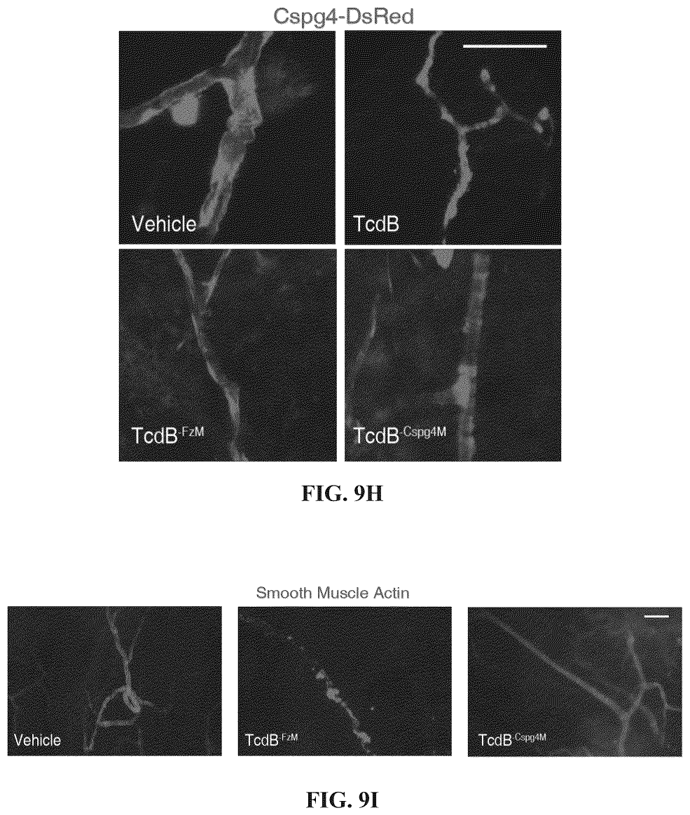

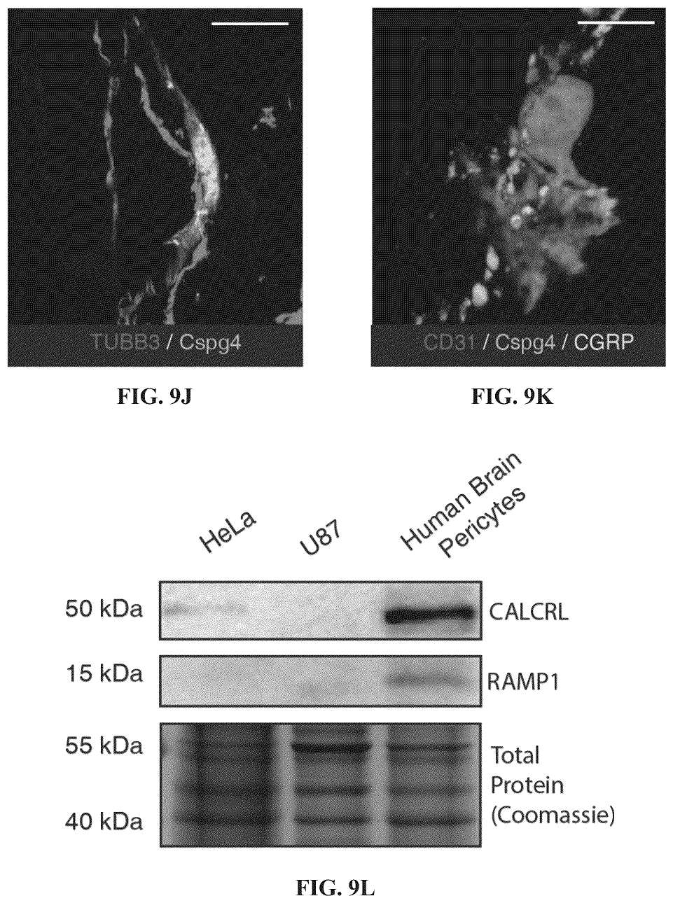

Unlike FZD1/2/7, CSPG4 is not expressed by neurons and was previously considered to be expressed by colonic myofibroblasts. Recent scRNA-seq of human colon tissues, however, suggests that CSPG4 expression is highly enriched in vascular mural cells including pericytes and vascular smooth muscle cells (FIG. 2D). Pericytes are contractile cells that wrap around microvascular endothelial cells and are well known for forming and regulating the blood-brain barrier. To identify Cspg4⁺ cells in the colon, Cspg4-DsRed transgenic mice were utilized, in which the Cspg4 promoter drives fluorescent DsRed reporter expression. Confocal imaging revealed two populations of DsRed⁺ cells, those that encircled large branching vessels and had morphologies typical of vascular smooth muscle cells (FIG. 2E), and a population with fine processes that wrapped around the subepithelial blood vessels in the colonic mucosa (FIGS. 2E-2F). These mucosal Cspg4⁺ cells always closely apposed vascular endothelial cells marked by CD31, but not lymphatic endothelial cells, and formed a perivascular network consistent with being pericytes (FIGS. 9A-9B). Mucosal Cspg4⁺ cells robustly expressed transcripts for pericyte marker platelet derived growth factor receptor beta (PDGFRβ) (FIG. 8E). They did not colocalize with markers for other intestinal cell types including enteric glia, interstitial cells of Cajal, mast cells, myofibroblasts/colonic smooth muscle cells, and stromal fibroblasts (FIGS. 9C-9F).

To test the necessity of CSPG4 for TcdB to target pericytes, isolated primary human brain pericytes were utilized as gut pericytes could not be cultured. TcdB was highly potent at inducing cell-rounding of pericytes (FIG. 2G, FIG. 9G). A TcdB mutant toxin deficient in binding to CSPG4 (TcdB-Cspg4M) showed markedly reduced potency, unlike TcdB-FzM (FIG. 2G). The double mutant (TcdB-FzCspg4M) showed similar potency as TcdB-Cspg4M (FIG. 2G). To validate whether TcdB targets pericytes via CSPG4 in vivo, an car injection model was utilized that allows imaging of pericytes surrounding car arterioles, which is difficult to do in gut tissues. Injection of TcdB or TcdB-FzM induced shrinkage of pericytes surrounding car arterioles, whereas injection of TcdB-Cspg4M showed no effect (FIGS. 9H-9I).

Pericytes are known to regulate vascular dilation and immune cell transmigration through changes in cell morphology and secretion of inflammatory cytokines. TcdB, but not TcdB-Cspg4M, induced interleukin 8 (IL-8) secretion from human brain pericytes (FIG. 2H). Consistently, exposure to TcdB in the cecal injection model increased the level of the cytokine CXCL1 (murine IL-8 homolog) in cecum tissues in vivo (FIG. 21). CXCL1 levels were not increased by TcdB-Cspg4M in WT mice or TcdB in Cspg4 KO mice (FIG. 2J).

Given that both neurons and pericytes are major regulators of vascular function, their ability to integrate signals was assessed. Immunohistochemical staining for the pan-neuronal marker TUBB3 showed an intermingled network of nerves and Csog4⁺ pericytes in the mucosa (FIG. 2K). Many of these nerve fibers were immunoreactive for CGRP, and CGRP⁺ varicosities closely associated with DsRed-labeled pericytes that wrapped around endothelial cells (FIG. 2L, FIGS. 9J-9K). Cultured human pericytes express the CGRP receptor components CALCRL and RAMPI (FIG. 9L) and adding CGRP to the culture medium induced secretion of IL-8 (FIG. 2M), confirming that pericytes can be regulated by CGRP.

### TcdB Action in a Footpad Model

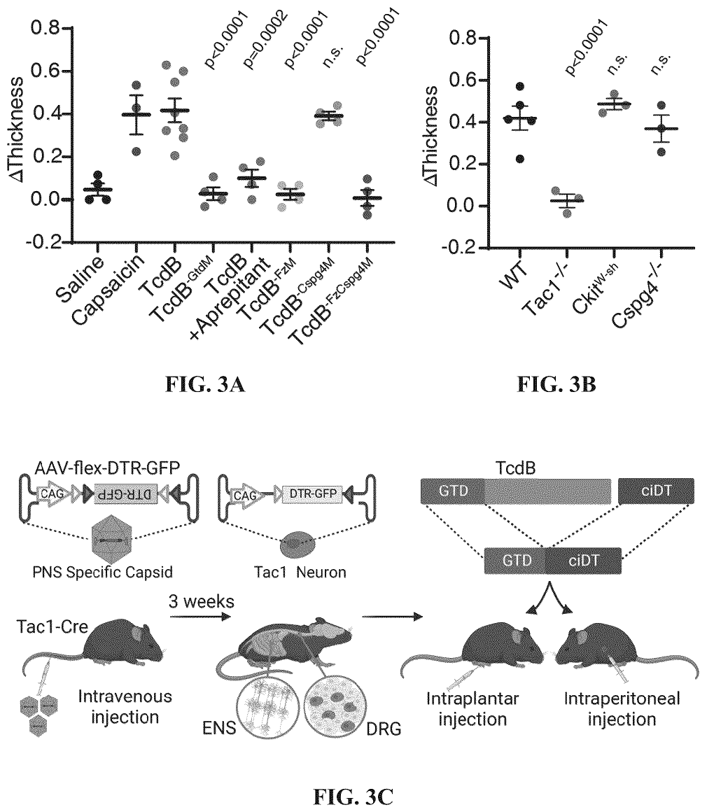

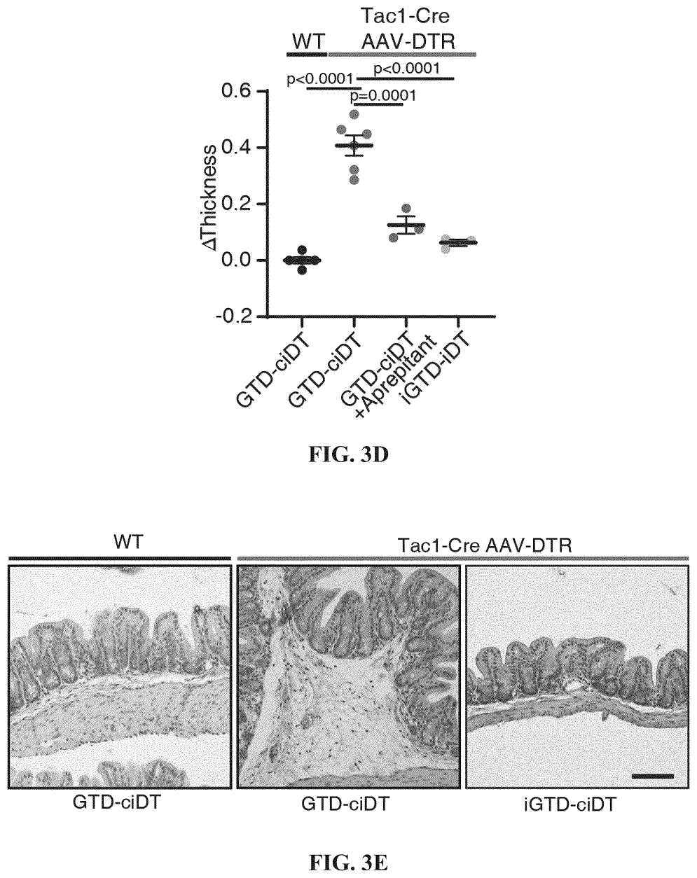

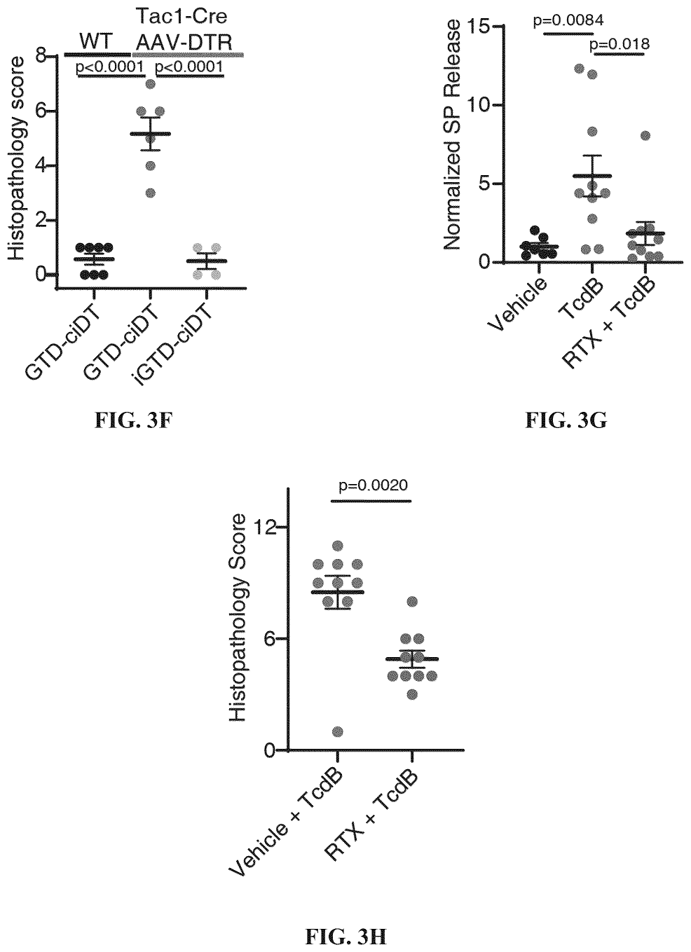

Neurogenic inflammation has been well studied in skin tissues, in which activation of peptidergic nociceptive sensory neurons drives rapid oedema and neutrophil infiltration. To further assess FZD-mediated targeting of sensory neurons by TcdB, a subcutaneous footpad injection mouse model was utilized, in which the occurrence of neurogenic inflammation can be assessed by measuring oedema (footpad thickness). Injection of TcdB in this model induced rapid development of oedema within 15 min (FIG. 3A), similar to injection of capsaicin, which activates the transient receptor potential cation channel subfamily V member 1 (TRPV1) in nociceptive sensory neurons (FIG. 3A). Blocking SP signaling using an FDA-approved small molecule drug, aprepitant (an NK1R antagonist), prevented oedema induced by TcdB (FIG. 3A). Consistently, TcdB did not induce oedema in Tac1 KO mice (FIG. 3B). Mast-cell-mediated allergic reactions may also induce rapid oedema. TcdB, however, induced oedema in mast-cell-depleted mice (c-Kit^{W-sh} mice) similar to WT mice (FIG. 3B). TcdB-FzM did not induce oedema in WT mice whereas TcdB-Cspg4M was as potent as TcdB (FIG. 3A). Furthermore, TcdB induced similar levels of oedema in Cspg4 KO mice and WT mice in this footpad injection model (FIG. 3B). These findings suggest that targeting FZD1/2/7 is sufficient to induce neurogenic inflammation, consistent with a model in which sensory neurons act upstream of pericytes.

### Delivery of TcdB-GTD Into Neurons Induces Inflammation

To determine if TcdB-GTD enzymatic activity in neurons is sufficient to induce neurogenic inflammation, a targeted effector delivery approach was developed, termed toxogenetics (FIG. 3C). This approach is similar to widely utilized selective expression of simian diphtheria toxin receptor (DTR) in mouse models for acute ablation of DTR-expressing cells through injection of DT,. This approach incorporates two changes: (1) DTR expression (limited by a LoxP-flanked stop cassette) is encoded and delivered using the adeno-associated virus (AAV) serotype PHP.s, which is highly selective toward peripheral neurons. When injected into Tac1-Cre mice, DTR would be expressed only in Tac1-positive neurons; (2) a detoxified DT mutant containing three-point mutations that deactivate its enzymatic activity, termed catalytically inactive DT (ciDT), was utilized as a carrier to deliver a fused cargo protein into DTR-positive cells in vivo (FIG. 3C).

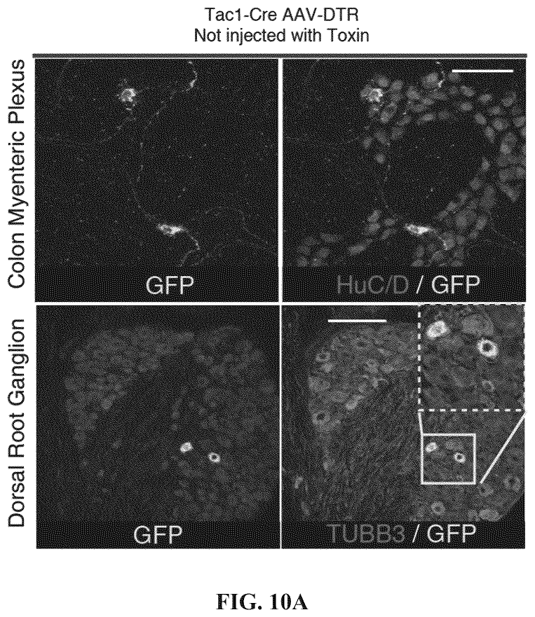

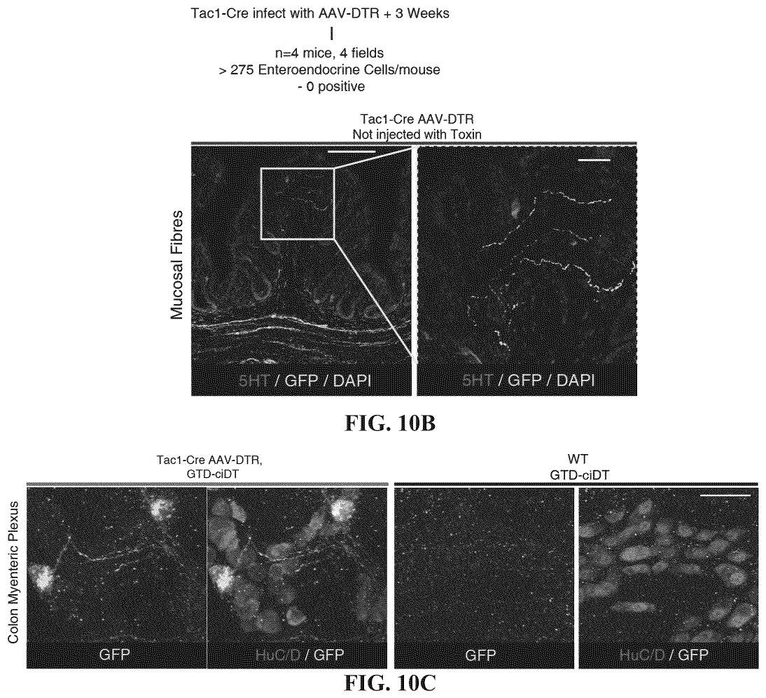

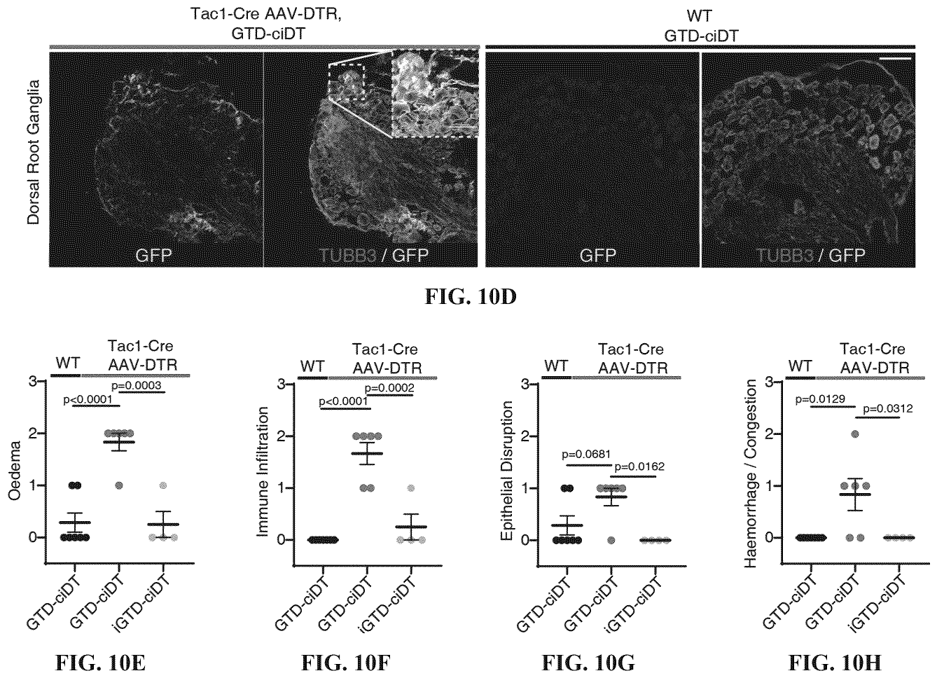

Injection of AAV-DTR at low titers (˜10¹¹) into Tac1-Cre mice led to expression of DTR in DRG and ENS neurons, but not non-neuronal Tac1⁺ cells such as enterochromaffin cells (FIGS. 10A-10D). A fusion protein was constructed composed of the TcdB-GTD domain fused with ciDT (GTD-ciDT). Injection of GTD-ciDT into the foot pads of uninfected control mice did not result in oedema, whereas injection into Tac1-Cre mice infected with AAV-DTR induced rapid development of oedema that was blocked by aprepitant (FIG. 3D). The enzymatic activity of the GTD can be inactivated through a point mutation (D270N, iGTD). Administration of iGTD-ciDT fusion protein did not induce oedema in Tac1-Cre mice infected with AAV-DTR (FIG. 3D).

Systemic administration of TcdB via intraperitoneal (IP) injection can cause death of mice. In contrast, injection of GTD-ciDT did not affect viability, likely because GTD-ciDT targets only Tac1⁺ cells. This allowed for the administration of GTD-ciDT systemically, which caused diarrhea within 3 h in Tac1-Cre mice infected with AAV-DTR. Histopathological analysis revealed severe oedema as well as mild neutrophil infiltration and epithelium disruption in colonic tissues (FIGS. 3E-3F, FIGS. 10E-10H). In contrast, neither administration of GTD-ciDT into control mice uninfected with AAV-DTR nor administration of inactive iGTD-ciDT into Tac1-Cre mice infected with AAV-DTR resulted in any diarrhea or damage to colonic tissues (FIGS. 3E-3F). These findings demonstrate that targeted delivery of the GTD domain into peripheral Tac1⁺ neurons alone is sufficient to replicate major histopathological changes associated with CDI.

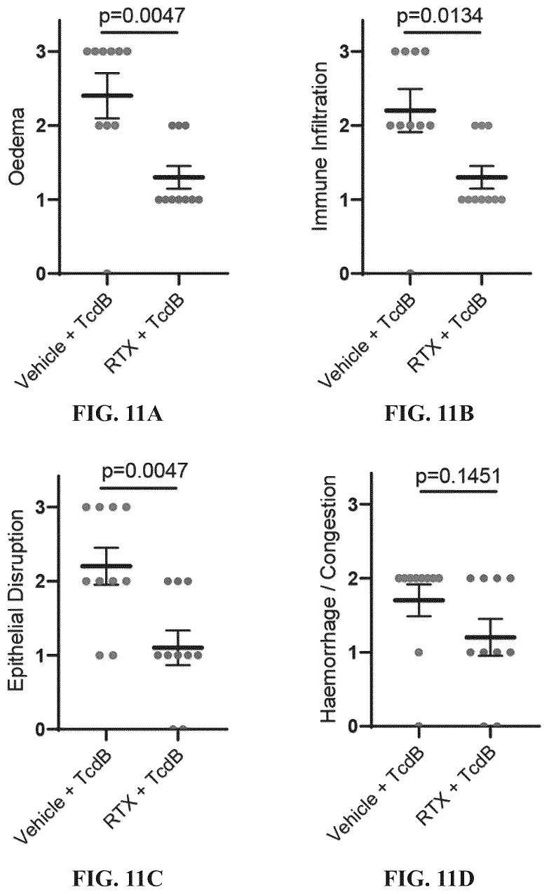

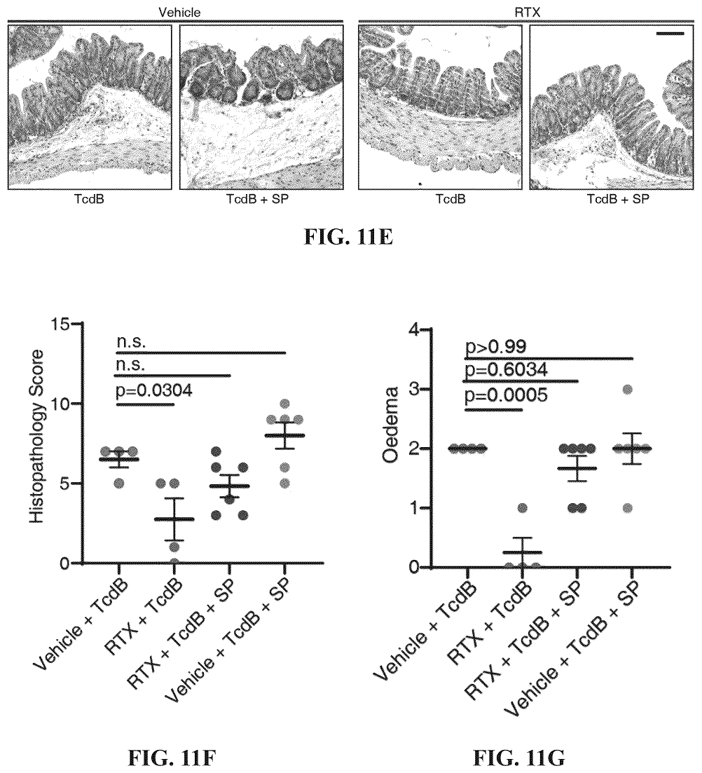

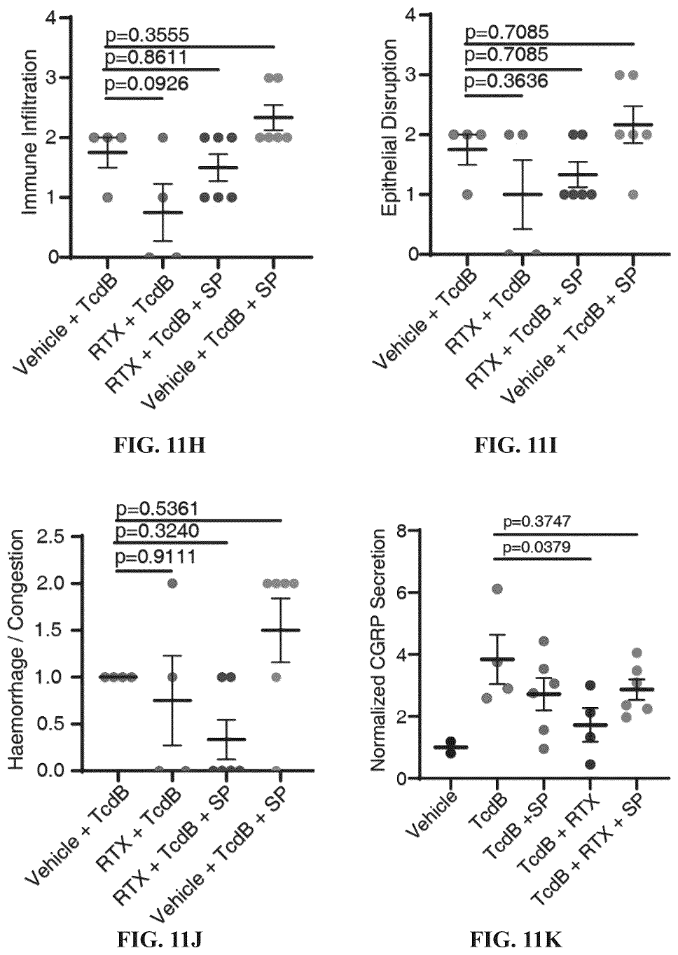

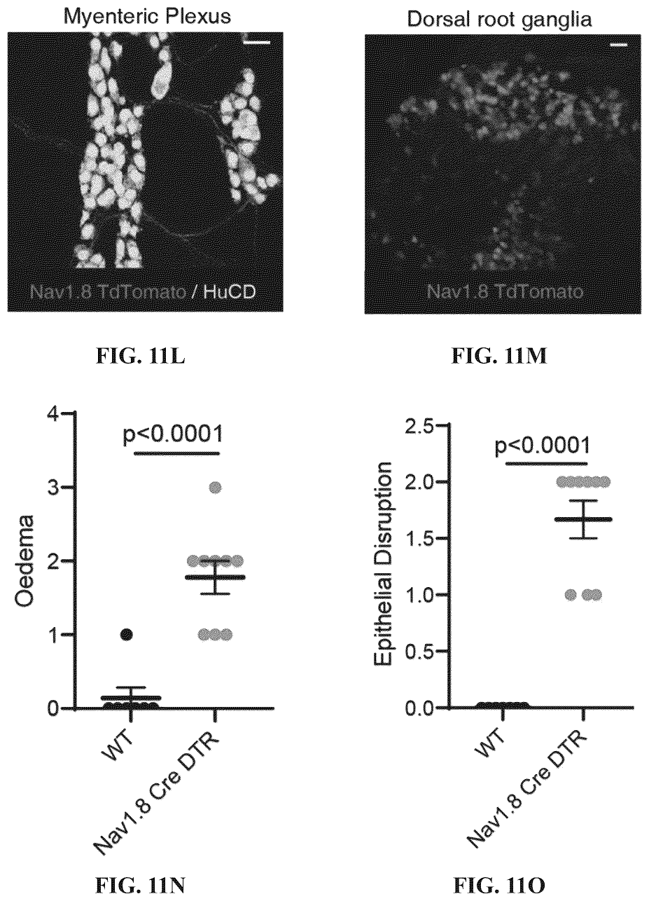

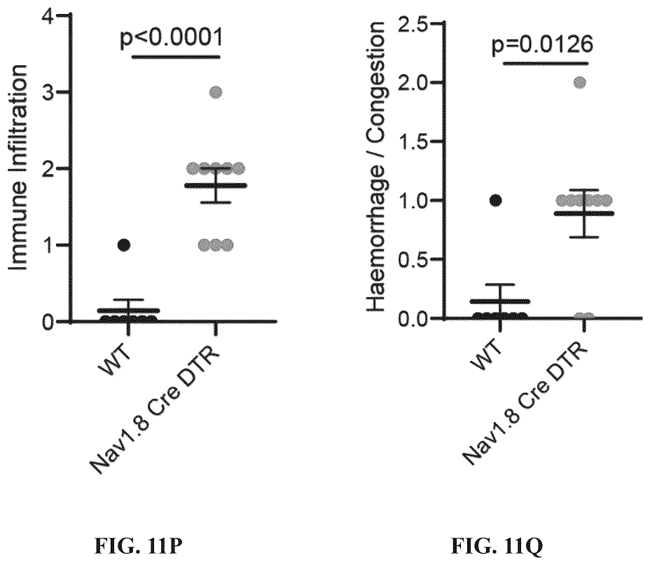

To determine the relative contributions of intrinsic and extrinsic Tac1⁺ neurons, two complementary approaches were taken. First, cecal TcdB injections were performed in mice pre-treated mice with resiniferatoxin (RTX), a strong TRPVI agonist that causes depletion of TRPV1⁺ sensory nerve terminals. RTX-treated mice showed reduced SP levels as well as less severe histopathology upon TcdB administration compared to vehicle-treated controls (FIGS. 3G-3H, FIGS. 11A-11D). Co-administration of SP with TcdB to RTX-treated mice restored histopathological scores (FIGS. 11E-11K).

Second, it was previously shown that sodium channel Nav1.8 (Scn10a) is selectively expressed in gut-innervating extrinsic sensory neurons, but not enteric neurons in the small intestine. This was confirmed to be the case in the colon using Scn10a^{Cre/+} Rosa25^{LSL-tdTomato/+} mice (FIGS. 11I-11M). By crossing Scn10a^{Cre/+} mice with Rosa-LSL-DTR mice, mice with DTR selectively expressed in extrinsic sensory neurons but not enteric neurons were obtained. Injection of GTD-ciDT into Nav1.8-Cre-DTR mice induced histopathological changes typical of CDI (FIGS. 3I-3J, FIGS. 11N-11Q). These data suggest that targeting extrinsic sensory neurons is sufficient to elicit neurogenic inflammation in the colon and recapitulates major elements of CDI pathogenesis.

Blocking Inflammation Reduces C. difficile Colonization

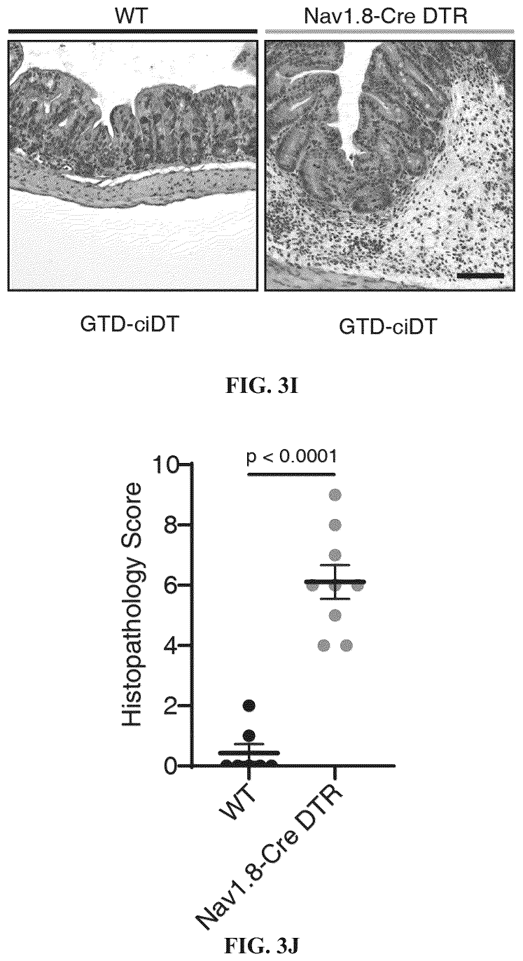

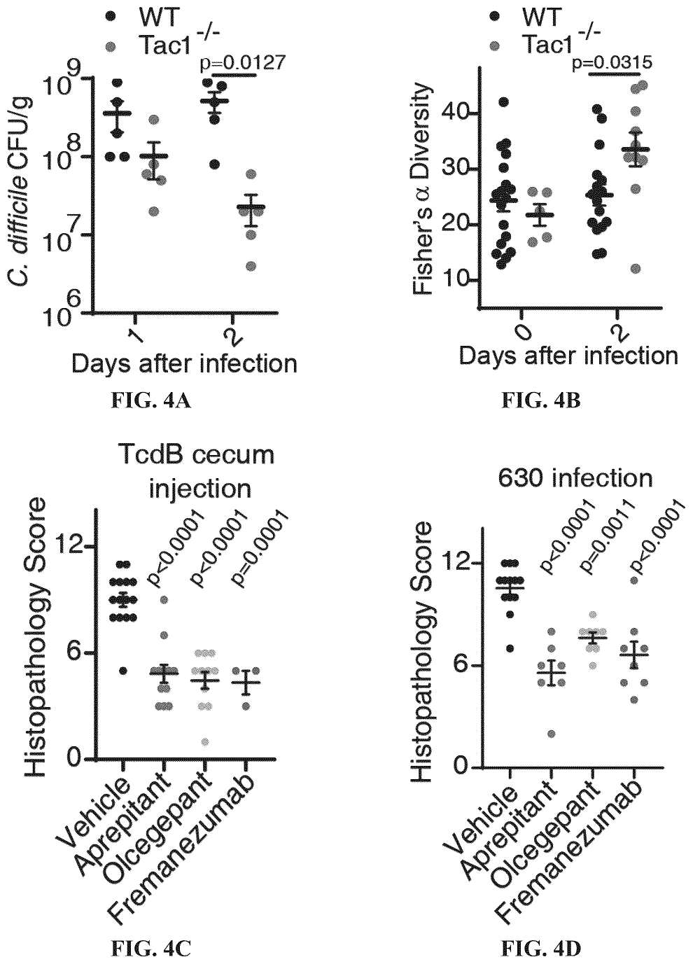

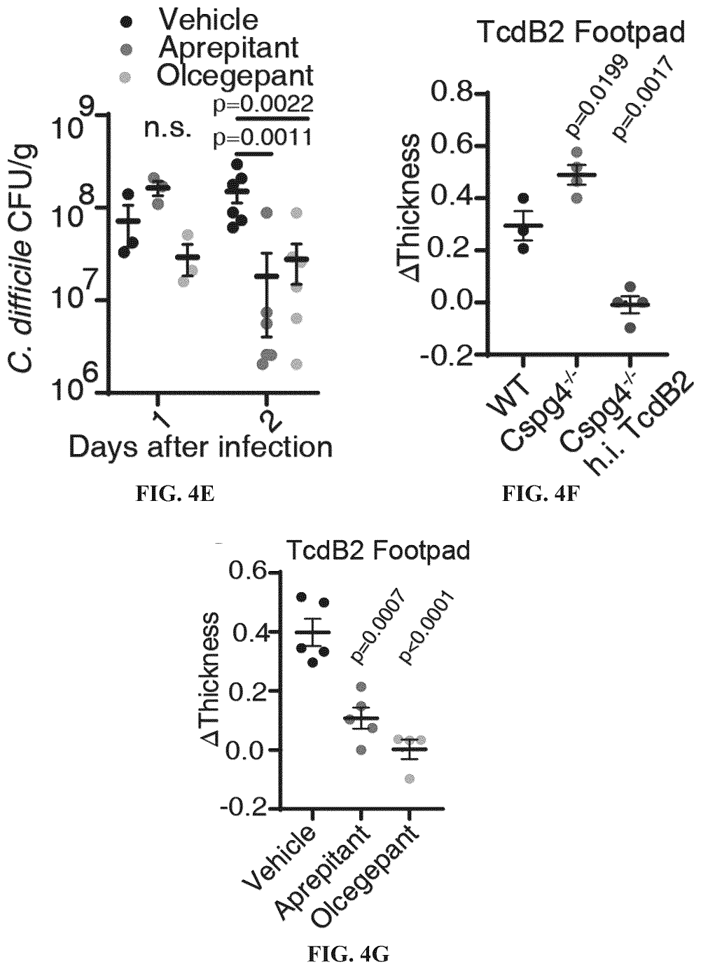

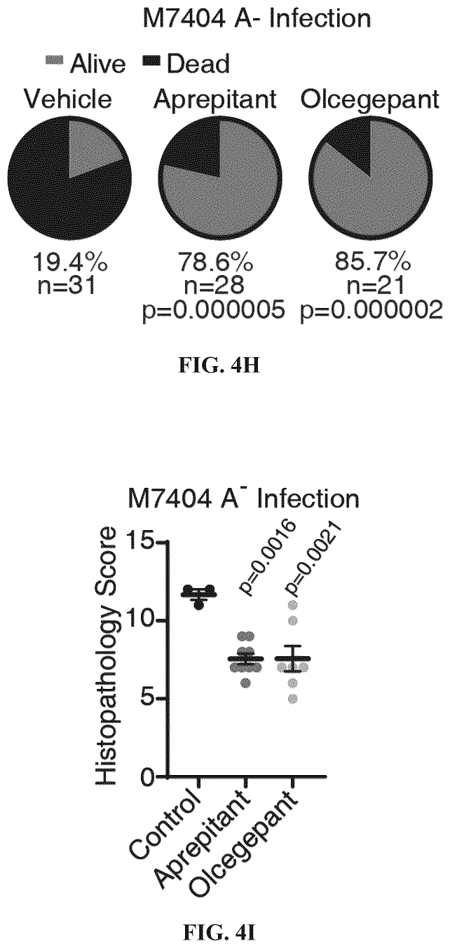

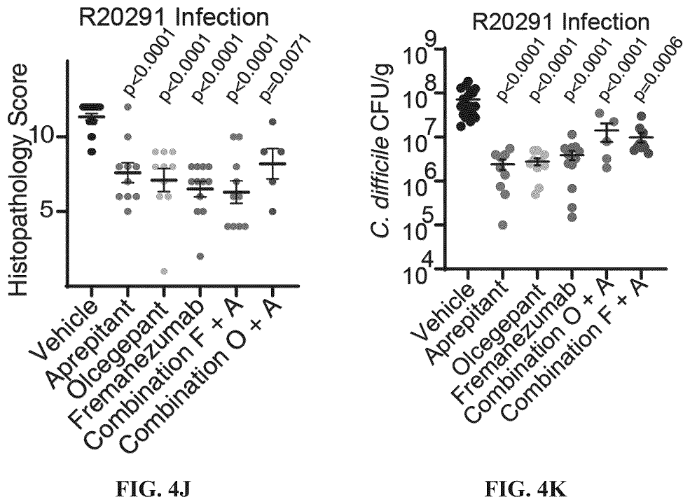

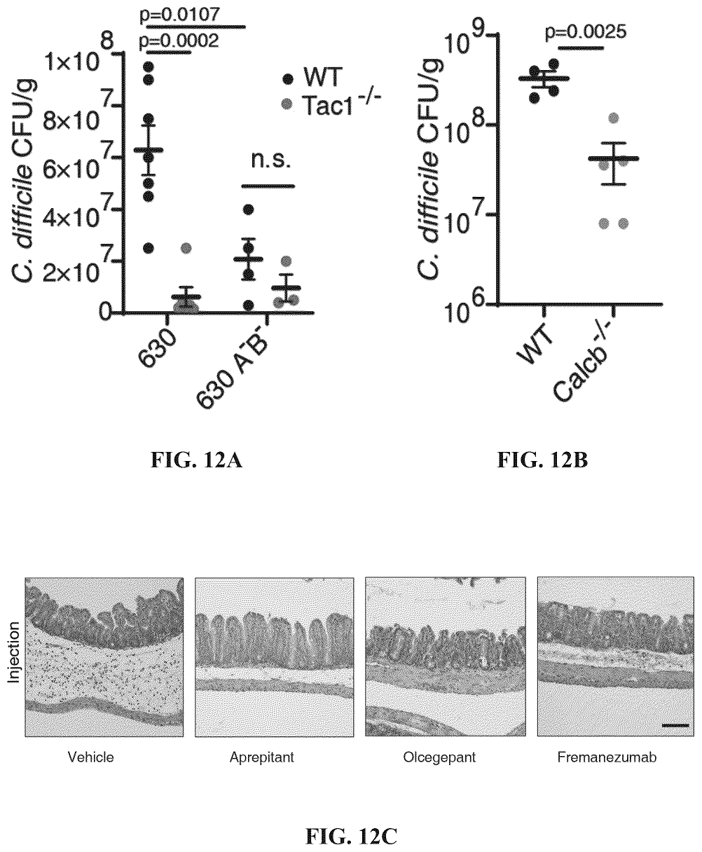

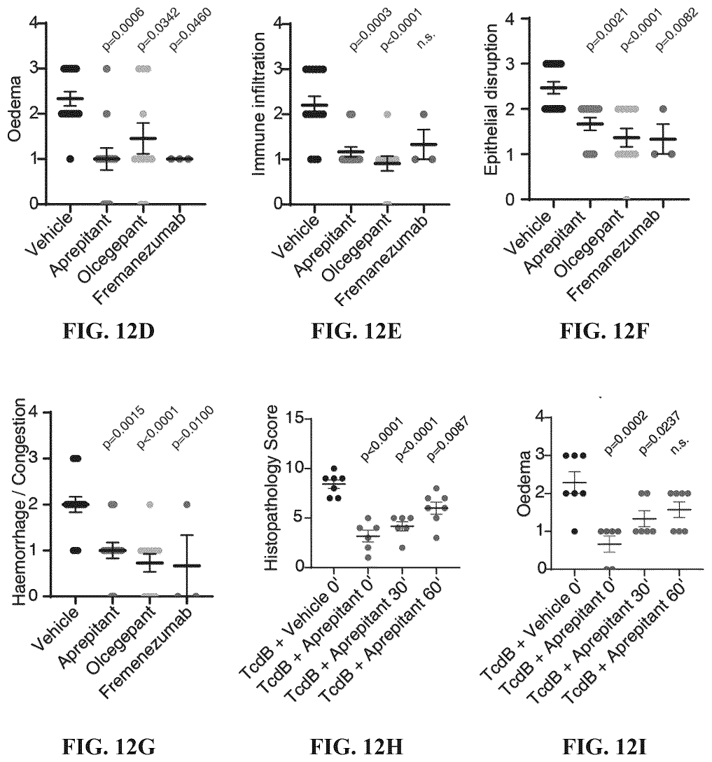

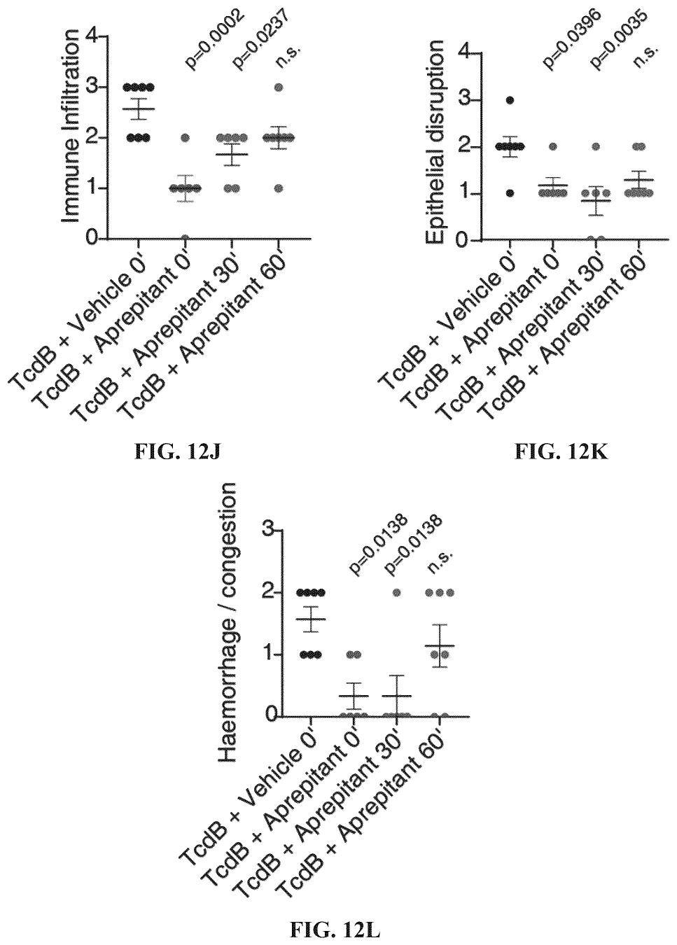

Recent studies showed that toxin-induced inflammation promotes C. difficile colonization. Consistently, although C. difficile burden measured by colony-forming units (CFUs) on the first day of infection was at similar levels between WT and Tac1 KO mice, Tac1 KO mice showed a greatly reduced C. difficile burden by day 2 (FIG. 4A). This reduced pathogen load was at similar levels to a mutant C. difficile lacking TcdA and TcdB administered to WT and Tac1 KO mice (FIG. 12A). Similarly, C. difficile burden was reduced on day 2 in Calcb KO mice compared with WT mice (FIG. 12B).

It has been reported that toxin-mediated inflammation suppresses the recovery of microbiome diversity, thus maintaining a non-normative state favorable for CDI. Consistently, recovery of microbiome community diversity, measured by 16S ribosomal RNA gene sequencing and quantified by Fisher's α diversity index, is suppressed in WT mice between days 1 and 2 of CDI, whereas microbiome diversity begins to increase on day 2 in Tac1 KO mice (FIG. 4B). Thus, depletion of inflammatory neuropeptides not only directly reduces damage to colonic tissues during CDI, but also decreases C. difficile colonization and promotes recovery of microbiome diversity.

### SP or CGRP Blockade Alleviates Infection

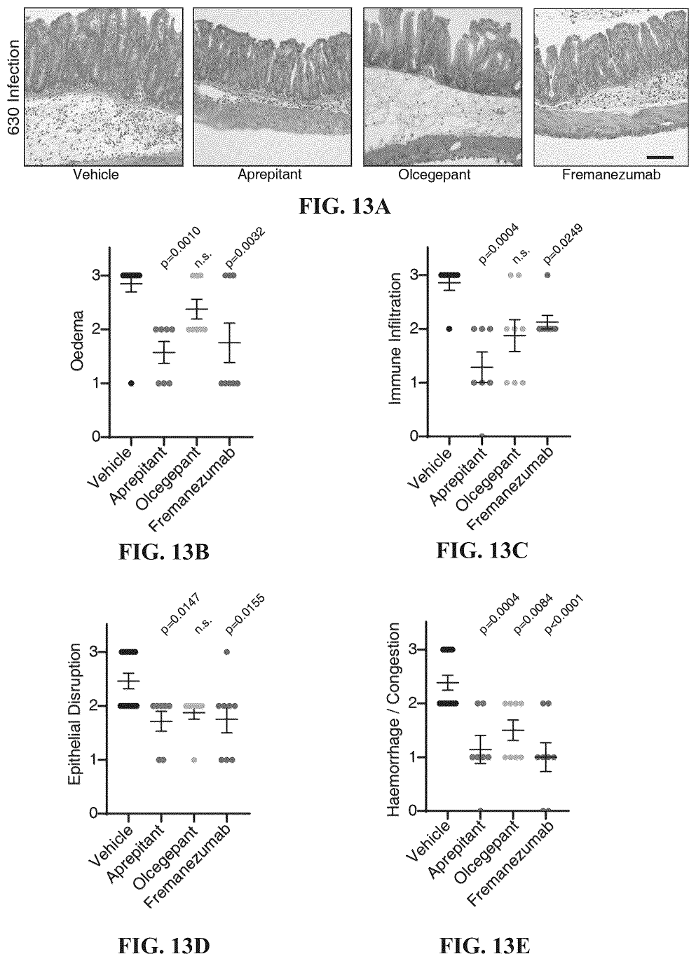

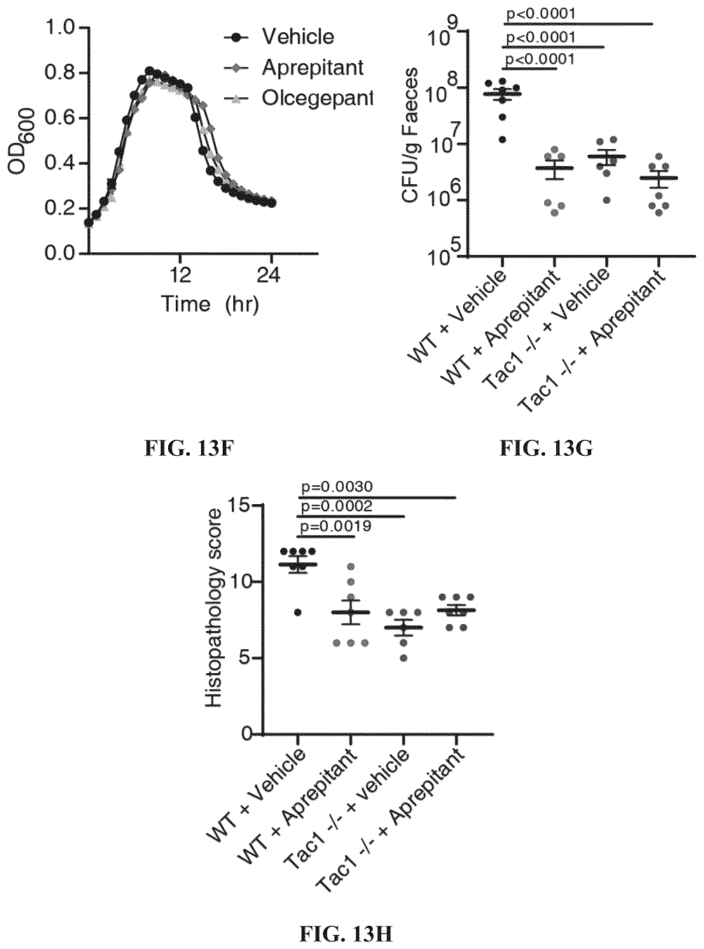

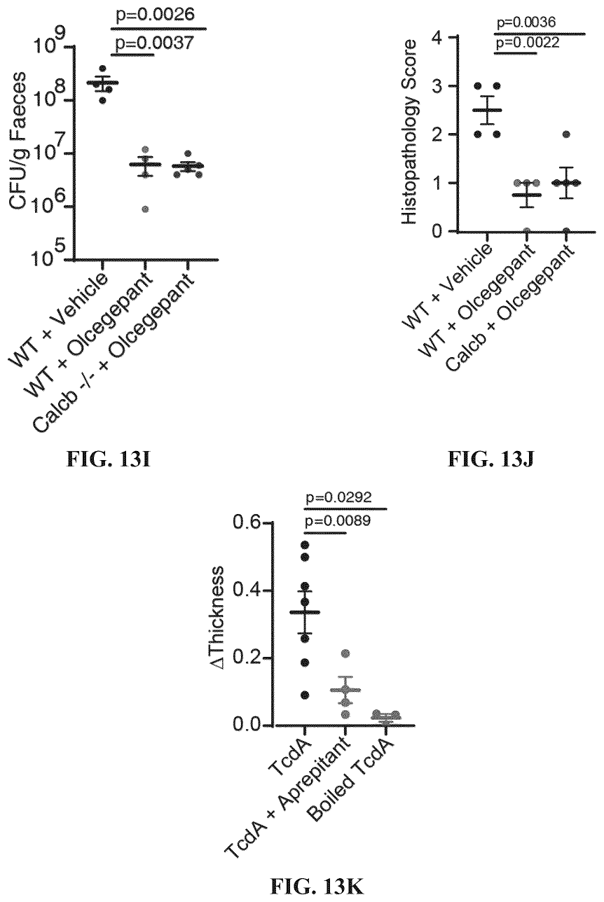

SP signaling can be blocked using aprepitant, an FDA-approved agent for treating nausea and vomiting. CGRP signaling can be inhibited with either small molecule CGRP receptor antagonists, such as olcegepant, or FDA-approved monoclonal antibodies that bind CGRP, such as fremanczumab, which are used for treating migraine headaches. Administration of aprepitant, olcegepant, or fremanczumab reduced histopathological scores in both cecum injection assays with TcdB and in CDI mouse models using C. difficile 630 Δerm (FIGS. 4C-4D, FIGS. 12C-12L and FIGS. 9A-9E). In mice with CDI, both aprepitant and olcegepant reduced pathogen burden on day 2 after infection (FIG. 4E). Neither drug influenced C. difficile growth rate in vitro nor had an effect in Tac1 or Calcb KO mice, respectively (FIGS. 13F-13J), suggesting that reduced pathogen colonization in CDI is not due to a direct effect on C. difficile.

A major challenge for treating CDI is the emergence of hypervirulent strains such as ribotype 027. These strains often encode a major variant of TcdB classified as TedB2, which contains ˜8% sequence variation from the standard TcdB in C. difficile 630 Δerm and does not bind to FZD1/2/7. It was found that injection of TedB2 into mouse footpads induced oedema, which was prevented by aprepitant or olcegepant (FIG. 4F), indicating that TedB2 also induces neurogenic inflammation. The effect is independent of Cspg4, as TedB2 induced footpad oedema in Cspg4 KO mice (FIG. 4G). Furthermore, TcdA also induced oedema in footpad injection assays, which was blocked by aprepitant (FIG. 13K).

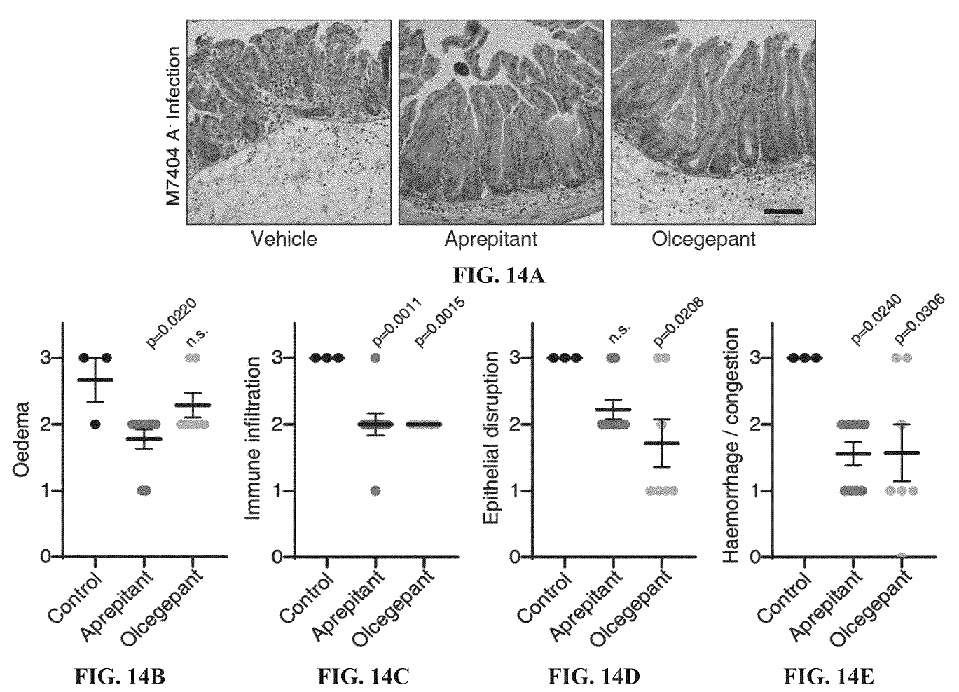

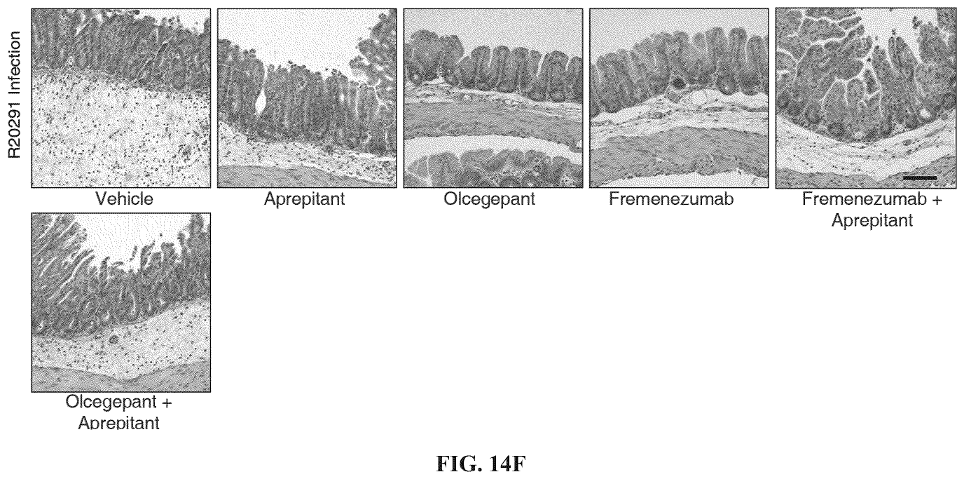

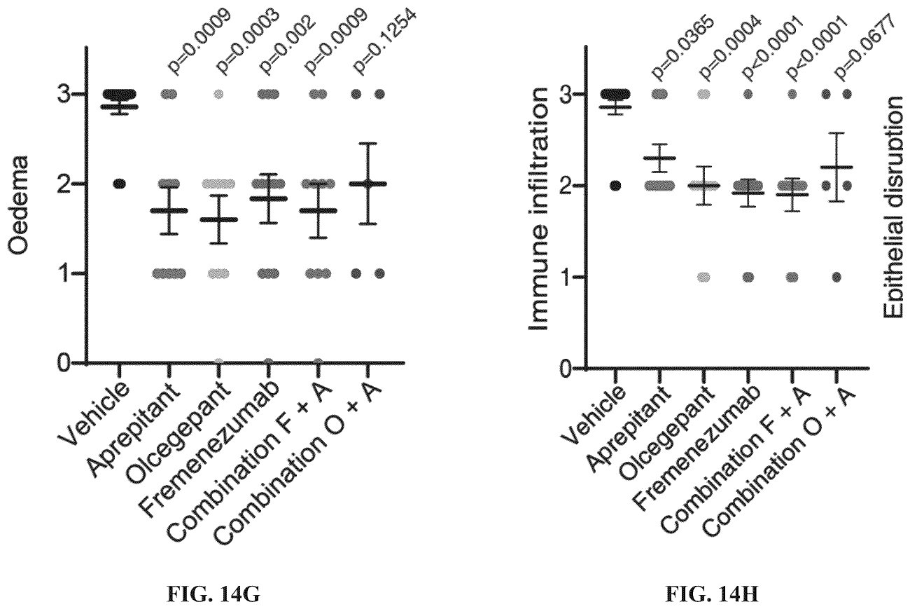

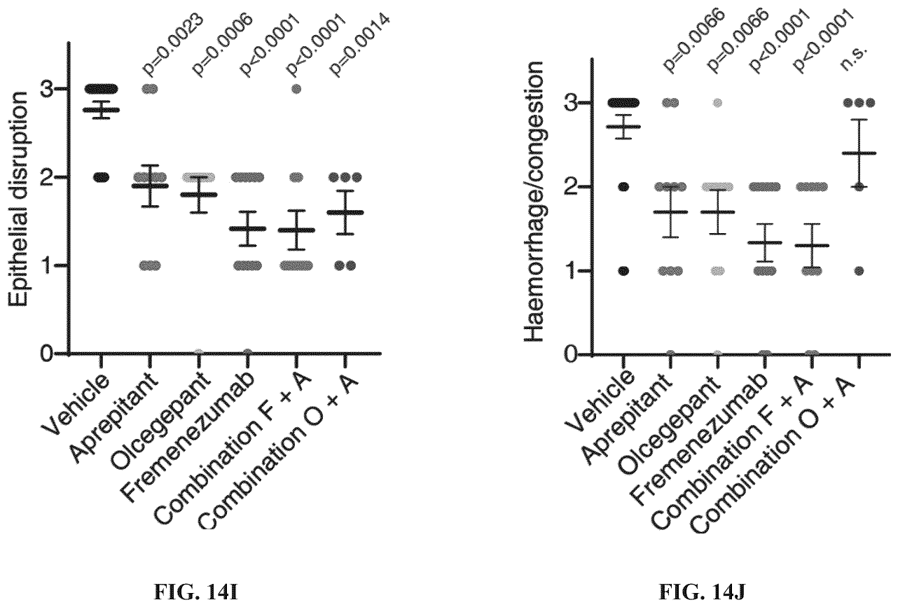

A genetically modified C. difficile strain was then utilized, that expresses TcdB2, but not TcdA (M7404 A-/B+), and is known to be hypervirulent, causing death in the majority of mice in a CDI model (FIG. 4H). Treatment with aprepitant or olcegepant markedly reduced fatality as well as histopathological scores of colitis in surviving mice (FIGS. 4H-4I, FIGS. 14A-14E). Therapeutic effects against a WT hypervirulent strain R20291 that expresses both TcdA and TcdB2 was further examined. Treatment with aprepitant, olcegepant, or fremanezumab all reduced histopathological scores in colonic tissues in CDI models (FIG. 4J, FIGS. 14F-14J), and reduced colonization of R20291 (FIG. 4K). Combinations of aprepitant and olcegepant or aprepitant and fremanezumab did not show additive effects over monotherapy (FIGS. 4J-4K, FIG. 14F-14J).

### DISCUSSION

It is established herein that TcdB induces neurogenic inflammation by targeting gut-innervating peptidergic neurons and pericytes in mouse models. The specificity of TcdB is mediated by its receptors FZD1/2/7, which are enriched in ENS and DRG neurons, and CSPG4, which is a marker for pericytes. The ability to induce neurogenic inflammation is shared by TcdB2 and TcdA, reflecting a central role of neurogenic inflammation in CDI. TcdB2 and TcdA do not utilize FZD1/2/7 as receptors and alternative potential receptors have been suggested, although the molecular basis for their targeting of neurons remains to be established.

Using the toxogenetic approach disclosed herein, it was demonstrated that TcdB in Tac1⁺ neurons alone was sufficient to induce neurogenic inflammation and recapitulate major histopathologic changes associated with CDI. It is noted that expression of FZD1/2/7 is not restricted to neurons and the pro-inflammatory effects of TcdB are likely multifaceted including damage to colonic epithelium, stem cells, and immune cells. Additionally, tissue damage may generate an inflammatory environment that activates sensory neurons and further promotes neurogenic inflammation.

Lastly, it is presented herein that targeting neurogenic inflammation with agents blocking SP and CGRP is an effective, host-oriented therapeutic approach in rodent models of CDI. These agents have already proven safe and effective in other clinical contexts. Clinical trials are needed to determine if these agents might have efficacy in treating human CDI.

### Methods

### MOUSE MODELS

All experiments were performed in accordance with animal protocols approved by the at Boston Children's Hospital Institutional Animal Care and Use Committee. Animals were housed in a specific pathogen free facility, except for infection experiments which were conducted in a facility that contains specific pathogens (ABSL2+). Animals were housed under standard conditions with ad libitum food and water, 22° C.+/−2C, 35-70% humidity, and 12-hour light-dark cycle. WT mice were from Envigo (CD1, Female 18-20 g), Jackson Lab (C57/BL6J, JAX: 000664), or bred in the colony (C57/BL6N, JAX: 005304). Calcb^{eGFP/+} (kindly provided by Ron Emeson, Vanderbilt University), Calca^{CreERT2/+} (kindly provided by Pao-Tien Chuang, UCSF), Cspg4^{-DsRed}(JAX: 008241), Tac1-Cre (JAX: 021877), Tac1^{-/-} (JAX: 004103), Cspg4^{-/-} (kindly provided by William Stallcup), Nk1r^{CreERT2} (JAX: 035046), Nav1.8-cre (JAX: 036564), Rosa-LSL-DTR (JAX: 007900), Rosa-LSL-TdTomato (JAX: 007909), c-kit^{W-sh} (JAX: 030764), and PLP1-EGFP (JAX033357) mice, have all been previously described. Calcb^{eGFP/eGFP} mice are referred to as Calcb KO, Calca^{CreERT2/CreERT2} as Calca KO, and NK-1r^{CreERT2/CreERT2} as Nk1lr KO. Tac1 KO, c-kit^{W-sh}, and Cspg4 KO were backcrossed extensively, maintained as homozygous colonies, and compared to C57/BL6J mice raised at the same time. Tac1-Cre and Nav1.8-Cre DTR were compared to C57/BL6J. Calca KO and Calcb KO were compared to littermate controls. Nk1r KO mice were compared to C57/B16NJ mice given that they were raised on this background. Mice were co-housed where littermate controls were used and across all pharmacological experiments. Tac1 KO and NK1R KO mice were not cohoused routinely, except that Tac1 KO mice for the experiments described in FIG. 13G-H were cohoused with their WT control mice from weaning.

### CECUM INJECTION ASSAYS

Cecum injection assays followed a previously established protocol. Mice were fasted 12-16 h prior to experiments and anaesthetized by isoflurane (2-4%) with oxygen (0.8 L/min). The cecum was exposed using a midline laparotomy and 6 μg of toxins (TcdB, TcdB-FzM, TcdB-Cspg4M or vehicle control) was injected into the lumen in 100 μl of normal saline. Internal fascia were then closed with absorbable sutures (Johnson and Johnson, Vicryl 4/0 RC) and overlying skin was closed using wound clips. Following surgery, all mice were given buprenorphine (Ethiqa XR, Fidelis) analgesia (1 mg/kg) by subcutaneous administration then allowed to recover. After the indicated incubation periods (6 h unless otherwise stated in the Brief Description), mice were euthanized by CO₂ inhalation, and the colon was dissected, flushed with PBS, and processed for downstream experiments. In experiments involving transgenic mice, both male and female mice were used with their respective matched genetic controls, which did not display sexually dimorphic responses. For pharmacological assays, only female CDI mice were used for cecum injection assays. CDI mice were injected with pharmacological agents 30 min to 1 h prior to cecum injection. Aprepitant (100 mg/kg, Tocris, 6486) and olcegepant (5 mg/kg, Medchemexpress, HY-10095) were dissolved in DMSO and diluted into 2% Tween-80 in normal saline and administered to mice by intraperitoneal (IP) injection. Fremanczumab (100 mg/kg, Ajovy, 225 mg One-Prefilled Syringe, obtained via Boston Children's Hospital Central Pharmacy (BCHP), NDC 51759-204-10) was dissolved in normal saline. For the rescue experiments TcdB was co-administered with 10 nmoles of SP, and mice were euthanized 2 h later.

### CDI INFECTION MODEL

A previously developed antibiotic-treatment-induced CDI mouse model was used. These experiments were performed in an A-BSL2 facility with autoclaved food (Prolab RMH 3500, Autoclavable), cages, and water. C difficile strains 630 Δerm, 630 Δerm (TcdA⁻/TcdB⁻), M7404 (TcdA⁻), and R20291 were used. A mixture of antibiotics including vancomycin (0.4 mg/mL, Sigma-Aldrich, A94747) colistin (850 U/mL, Sigma-Aldrich, C4461), metronidazole (0.215 mg/mL, Sigma-Aldrich, M1547), gentamicin (0.035 mg/mL, Research Products International, G38000), and kanamycin (0.045 mg/mL, Sigma-Aldrich, K1377) were diluted in drinking water and used to feed mice for 3 days, followed by regular water for 2 more days, then mice were injected with clindamycin 10 mg/kg, IP, Mylan pharmaceuticals, obtained from BCHP, NDC 67457-816-00) in normal saline. Twenty-four h later, mice were gavaged with 10⁴ spores diluted in PBS. Mice were monitored twice daily and euthanized if they became moribund (trouble breathing, >15% body weight loss).

For experiments utilizing transgenic mice: 8-15-weck-old mice were infected with C. difficile 630 Δerm, their tissues were collected two days after infection and analyzed for histopathology. Both male and female mice were used. For simplicity, the results of WT mice that reflect multiple genetic backgrounds are combined in FIGS. 1A-4J (they are presented separately in FIGS. 5A-14J). Pharmacological assays for CDI infection were performed in WT C57/BL6J mice. Mice were injected daily with the small molecule antagonists. Fremanczumab was injected at the time of spore gavage. In experiments involving R20291, mice were injected with fremanezumab prior to the experiments at the same time clindamycin was injected.

### HISTOLOGY ANALYSIS AND SCORING

For histological analyses, tissues were trimmed, placed in neutral buffered formalin (10%), and then transferred to PBS at 4° C. prior to embedding by the Beth Israel Deaconess Medical Center (BIDMC) core facility. Blocks were cut at 6 μm and subjected to haemotoxylin and cosin (H&E) staining. Briefly, slides were baked, deparaffinized in xylene, re-hydrated with serial ethanol washes, immersed in Gill's haemotoxylin (Number 3, Thermo Fisher, Shandon), washed, and then blued with Scott's Tap Water (0.1% W/V sodium bicarbonate in water) prior to immersion in acidic eosin. Samples were dehydrated through serial changes of ethanol and then xylene prior to mounting in DPX. Slides were then left to dry overnight in a fume hood. Sections were imaged and scored blindly according to standardized criteria (oedema, epithelial disruption, immune infiltration, and haemorrhage or vascular congestion), scored from 0-3 (0-Normal, 1-Mild, 2-Moderate or 3-Severe, see FIGS. 5A-5E). Only animals that were euthanized at the indicated endpoint were included in scoring. De-identified images of sample histopathology from human CDI compared to healthy colon were provided by Dr. Jeffrey Goldsmith. Sample images were from cases that had been previously collected, processed, sectioned and H&E stained as part of routine patient care at Boston Children's Hospital and Brigham & Women's Hospital.

### SINGLE CELL DATA ANALYSIS

Single cell data were extracted from published datasets. For analysis of human and mouse Frizzled expression in colonic cells, the data was extracted from a comprehensive atlas of mouse/human enteric neurons using the Broad institute single cell expression platform, normalized the data in Excel (data were normalized to the maximum expression), and generated plots shown in Tableau software (v2022.2). For analysis of DRG neuron expression of Frizzled in colonic innervating DRG neurons, data was extracted from the author's analysis tool. For the in situ hybridization dataset, spinal cord images were extracted from P4 mice from the Allen Brain atlas. For analysis of CSPG4 expression in human colon, the EMBL single cell analysis platform was used to visualize expression in a published dataset.

### IMMUNOHISTOCHEMISTRY

For analyses of Cspg4-dsRed, Nav1.8-TdTomato, and AAV-injected mice, intestines and/or DRG were isolated, and drop fixed in 4% paraformaldehyde (PFA) in phosphate-buffered saline (PBS) pH 7.4. For AAV-injected and DT-treated mice, mice were euthanized with CO2inhalation, a thoracotomy was performed to expose the heart, and then 15 ml of PBS was perfused followed by PFA through a 21G winged catheter. Following tissue isolation and fixation, intestines or DRG were processed for whole mount staining or cryoprotected using 30% sucrose. DRG were exposed by spinal laminectomy and isolated from mice by microdissection.

Whole mount staining was performed as previously described. Briefly, tissues were rinsed six times with PBS+0.5% Triton X-100 followed by incubation in blocking solution (PBS+5% normal goat or donkey serum+0.5% Triton X-100+20% DMSO) with diluted primary antibody for 48-72 h at room temperature (RT). Tissues were then rinsed and incubated overnight in blocking solution with secondary antibody at RT. Tissues were rinsed again with PBS+0.5% Triton X-100 six times and then mounted with Vectashield mounting media. For some samples, tissues were subjected to optical tissue clearing (BABB solution protocol as described previously). As controls, for analyses involving Cspg4-DsRed littermate non-carrier mice not expressing DsRed were used; for analyses involving GFP mice that were not injected with virus were used. For all other routine staining, initial experiments used a section stained with only the secondary antibodies.

For cryosections, intestinal segments and DRG were equilibrated in 30% sucrose/PBS overnight at 4° C. and embedded in optimal cutting medium (OCT, Sakura). For the intestine, cross-sections of 40, 70, and 100 μm were collected onto slides. DRG were sectioned at 10-12 μm. Staining was performed as previously described. Briefly, slides were air dried for >20 minutes, equilibrated with PBS, and then incubated in blocking solution (PBS+5% normal goat or donkey serum+0.1% Triton X-100 +/−5% BSA for intestinal segments, PBS+2% BSA or 5% normal donkey serum+0.1% Triton X-100 for DRG) for one hour at RT, followed by incubation with primary antibody in blocking solution overnight at 4° C. Slides were rinsed three times in PBS followed by incubation in secondary antibody diluted in blocking solution for 60 min. Slides were then rinsed three times in PBS and mounted in Vectashield media for intestinal segments, and in Vectashield or fluoromount media for DRG. For nuclear staining, DAPI (1:500from 200 μg/ml concentrate) was either diluted in blocking solution during the secondary antibody incubation step or Vectashield/Fluoromount containing DAPI was utilized during tissue mounting. As controls, for analyses involving Cspg4-dsRed wild-type mice not expressing DsRed were used and stained in the same way; for analyses involving GFP mice not injected with virus were used. For all other routine staining initial experiments, a section stained with only the secondary antibodies was used.

RNAscope fluorescent in situ hybridization was carried out via the vendor-provided protocol for fixed-frozen tissue sample and preparation (Advanced Cell Diagnostics) with Pdgfrb probe (Cat. No. 411381) with the following modifications: Activated ribonucleoside vanadyl complexes (New England Biolabs Cat. No. S1402S) were included in PBS at 10 mM during tissue collection. The tissue was fixed in EM grade 4% PFA at 4° C. for 24 h, equilibrated in 30% sucrose solution in PBS, and embedded in OCT media. 8 μm cryo-sections of colon were then collected and used. Opal 690 dye (Akoya Biosciences, FP1497001KT) was used at 1:1500. DapB (bacterial transcript) probe (Cat.No. 200470) was used as a negative control. Immunohistochemistry and tissue mounting were then performed as outlined above.

### Footpad Injection Assay

The footpad toxin injection assay was performed as previously described. Briefly, mice were restrained in an incontinence pad and the footpad was held and subsequently injected using a 29.5G syringe into the plantar surface. Mice were observed for 15 minutes, and measurements of paw diameter were performed by digital caliper measurement from the plantar surface to the top of the foot. Paw diameter changes were assessed by calculating a ratio relative to the un-injected foot. For each test toxin, 500 ng were administered in a volume of 20 μl of normal saline. Pharmacological assays were performed in female CDI mice, with pre-treatment with drugs 30 min to 1 h prior to toxin injection. For experiments involving transgenic mice, experiments were performed in male and female mice (age matched, 8-15 weeks). Capsaicin was dissolved in ethanol and diluted into a 10% Tween solution; 1 μg of capsaicin was injected into the footpad.

### Ear Toxin Injection Assay

CDI mice were anesthetized with isoflurane (2-3%), injections were made intradermally into the car using a 29.5G syringe. Toxins (1 μg) were diluted in normal saline. Mice were monitored and then euthanized after two hours. Their cars were dissected, split down the midline using No.5 style forceps and then fixed with acetone for 1 min prior to being washed in PBS, blocked in 2% BSA+0.3% Triton X-100, and stained for 2 h in blocking buffer at RT with anti-smooth muscle actin (Sigma Aldrich, 1A4, 1:200) and then secondary antibody staining for 1 h at RT. Experiments were replicated in Cspg4-DsRed mice, for those experiments 4% paraformaldehyde in PBS (pH 7.4) was used as fixative in replication experiments because acetone disrupted the signal of the DsRed.

### DRG Neuron Culture

DRG cultures were based on a protocol previously established with minor modifications. DRG were isolated into ice-cold Neurobasal A media (Gibco, 21103049) by laminectomy from exsanguinated mice following euthanasia by CO2 inhalation. DRG were dissociated in collagenase (5 mg/mL, Sigma, 10103578001) plus dispase (1 mg/mL, Sigma, 4942078001) in Hank's Buffered Salt Saline containing calcium (HBSS, Gibco) for 70 min. The enzymatic digest was neutralized by 10% Fetal Bovine Serum (FBS) and then centrifuged to pellet the DRG cells. DRG cells were resuspended into 2 mL of complete neurobasal media A (Gibco, 10888022), with nerve growth factor complex 2.5s 50 ng/ml (Sigma Aldrich, N6009), B27 plus 1X (Thermo Fisher Scientfic, A3582801), glial-derived neurotrophic factor (2 ng/ml, Sigma Aldrich, SRP3239), penicillin/streptomycin 1X (Gibco, 21103049), and Glutamax (1mM, Gibco, 35050061). Neurons were triturated 20 times with a wide bore flame polished glass pipette, then 20 times with a narrow bore glass pipette. The cells underwent gradient purification through a 10% Bovine serum albumin (BSA) gradient (in PBS) and were overlaid on top of the BSA solution using a glass pipette. Gradient purification was performed at 400 g for 12 min. Following gradient purification, the pellet was retained and resuspended in 1 mL complete media. The cells were examined by microscopy and then plated at a density of ˜2000 neurons per well in a 96 well or scaled accordingly for larger wells. Plates were coated in Poly-D-Lysine (0.1 mg/mL, Sigma, P6407) for 2 h, washed three times with water, then incubated with Laminin (10 μg/mL in PBS, Sigma, 11243217001) for 60 min. Plates were washed with PBS and dried before plating. Cells were allowed to grow for 3-5 days prior to experiments.

Confocal images

Confocal z-stack images for figures were captured using the Zeiss LSM 880 using super resolution deconvolution laser scanning confocal microscopy mode using objectives ranging from 10× to 63× prior to Airy deconvolution using Zeiss Zen Black software. In some cases, microscope images were captured using a Zeiss LSM 770. Routine fluorescent images were captured using an Olympus IX83 using a Lambda DG4 light source (Sutter Instruments) connected to a Hammatsu Orca Fusion camera and an Olympus BX53 using a LED light source and collected using Olympus Cellsens. Following deconvolution, z-stack images were rendered into maximum intensity projections and images were adjusted for brightness and contrast in ImageJ or Zeiss Zen Black software. For supplementary videos, z-stack images were processed and rendered into 3D image videos using Aivia image analysis software (Aivia, Inc., Leica Microsystems.) Videos were edited and converted to.mov format using Wondershare Uniconverter™ 14 software.

### Measuring Release of SP and Cytokines

SP in cell culture medium for DRG neurons was measured using the well-established enzyme-linked immunosorbent assay (ELISA) with a kit following the manufacturer's instruction (Cayman chemical, 583751). CGRP (Cayman, 589001) and CXCLI (Biolegend, 22141) in cecal samples were measured using established kits and following manufacturer's instructions. SP/CGRP/CXCL1 release from cecum samples was measured after cecum injection assays (6 μg toxins, 6 h incubation). Briefly, cecum tissues were dissected and flushed with PBS, a section of cecum was taken and weighed, release was measured for 60 min by incubation with agitation (400 rpm), at 37° C. in DMEM (Gibco, 11965084) without supplements. Media were collected and release assays for SP were performed using the ELISA assay. Data were normalized internally to the controls collected in parallel. For the infection and time course experiments mice were treated as normal for infection and collected at the indicated time points before SP and CGRP release was measured from explants.

Primary human cultured brain vascular pericytes (ScienCell, #1200) were routinely cultured in special medium with supplement, FBS and penicillin streptomycin provided by the cell's vendor (ScienCell, #1201). Cells were subcultured when confluent and used up to passage 10. For secretion assays these were performed on early passage cells (passages 3-8). Cytokine secretion in cell culture medium was determined using ELISA based assays against IL-8 (Biolegend, 431504). The effect of CGRP on cultured pericytes was determined by incubating pericytes for 1 h with human CGRPB (Cayman, 24725).

### Cell Rounding Assay

Cells were seeded on 96 well plates, serially diluted toxins were applied in their respective medias and incubated for five hours. Images were obtained on an Olympus IX83, and an Olympus IX71. Round cells were determined by eye, and the proportions were plotted in Graphpad Prism.

### Immunoblot

Cultured pericytes, U87 and HeLa cells were collected using trypsin-EDTA (0.05%, ThermoFisher scientific). Cells were collected by centrifugation, the pellet was washed in PBS. Cells were lysed in Radio-Immunoprecipitation Assay Buffer (50 mM Tris 8.0, 0.1% Triton X-100, 0.5% sodium deoxycholate, 0.1% sodium dodecyl sulfate, 150 mM NaCl) with protease inhibitor cocktail, then cell lysates were clarified by centrifugation at 14,000 g for 10 min. Subsequently, 10 μL of sample were diluted in Laemmli buffer containing DTT, then boiled for 10 min at 95° C. Samples were loaded into a hand-cast, 12% acrylamide gel (Bio-Rad), proteins were separated by SDS-PAGE at 125V prior to transfer to a nitrocellulose membrane using a semi-dry transfer system (Bio-Rad, Trans-blot Turbo). Membranes were blocked in blocking buffer (3% BSA in Tris Buffered Saline, 0.1% Tween-20, TBS-T), then incubated overnight at 4° C. with anti-RAMP1 (1:500, Alomone; ARR-021), anti-CALCRL (1:1000, Sigma-Aldrich, HPA008070), or anti-CALCR (1:1000, Bioss; bs-1860R) diluted in blocking buffer, the membrane was washed three times in TBS-T, then incubated in secondary antibody for 60 min (Goat anti-Rabbit HRP, BioRad, 1:2000). Blots were then washed three times in TBS-T prior to development using SuperSignal Chemiluminescent Substrate (ThermoFisher Scientific, #34080) and imaging on a BioRad GelDoc.

### Protein Purification

BL21 were transformed with expression plasmids and plated on kanamycin LB plates. Single clones were selected, sub-cultured, and transferred to larger volume flasks in autoinduction media with agitation, on an incubated shaking platform (at 37° C.) until they reached OD600 of 0.8. The temperature was then dropped to 18° C. for 48 h. Bacteria were collected by centrifugation at 4000 g for 15 minutes. For His tagged proteins, bacteria were lysed in TBS (50 mM Tris-HCl, pH7.5, 150 mM NaCl, 0.05% Tween-20, and 1 mM phenylmethylsulfonyl fluoride (PMSF)), sonicated (3 sec/8 sec time of on/off for 5 min total sonication time), supernatants were collected after centrifugation and then loaded onto prewashed Ni-NTA resin containing column, washed with 70 column volumes of wash buffer (TBS, 0.05% Tween-20, 10 mM imidazole), then eluted with TBS with 200 mM imidazole, buffer exchanged into TBS.

Recombinant TcdB refers to TedB1 sequence from VPI10463, codon optimized from VPI10463 and purified from E. coli as previously described. Recombinant TedB2 refers to TcdB2 sequence from R20291, purified using B. megaterium. Recombinant TcdA refers to TcdA sequence from VPI10463, purified from B. megaterium. Recombinant TcdB mutants were all purified in E. coli. TcdB-FzM (also known as TcdB-GFE), bears mutations disrupting Frizzled binding (V1595G/N1596F/F1597E, plus deletion of L1598, Q1599, and S1600). TcdB-Cspg4M bears mutations disrupting Cspg4 binding (S567E/Y603G/D1812G). TcdB-FzCspg4M is a combinational mutant containing both the mutations disrupting Frizzled binding and those disrupting Cspg4 binding. TcdB-GtdM contains mutations disrupting Rho GTPase binding wherein residues G444-K452 were replaced with the corresponding residues in TcdB from C. difficile strain M68, 1445-S453.

### Plasmids

GTD-ciDT was constructed by fusion of the TcdB-GTD (TcdB 1-543, based on the sequence in VPI10463, codon optimized for E. coli expression) in frame with ciDT containing three-point mutations (K51E/G52E/E148K, inactivating the enzyme domain) into pET28a vector (Novagen, with a C-terminal His-tag) through Gibson Assembly (NEB, E2621). A linker region (EFGSGSGSGA) was added between the GTD and ciDT. iGTD-ciDT was generated by site directed mutagenesis introducing a D270N mutation in GTD, which disrupts manganese co-ordination as previously described for full length TcdB. All constructs were confirmed by Sanger sequencing (Azenta).

### Toxogenetic Experiments

AAV-PHP.s Flex DTR: GFP viral particles were prepared by the Boston Children's Hospital virus core facility, with titer information provided. Viral particles (1×10¹¹) were diluted in normal saline. Mice (4-8 weeks) were transduced through intravenous (IV) injection, into the tail vein, using a 29.5G syringe with a volume of 100 μL under BSL2 conditions in a biosafety cabinet. Mice were tested/examined 3 weeks after virus transduction. For IP injection of GTD-ciDT and iGTD-ciDT, 500 ng of protein was diluted in normal saline and injected into mice. Mice were observed for 3 h, followed by euthanization. Cecum tissues were collected, processed, and analyzed. For germline expression of DTR in extrinsic afferent neurons, Nav1.8-cre mice were crossed with Rosa-LSL-DTR mice to generate Nav1.8 Cre/+: Rosa-LSL-DTR/+ mice and analyzed at 8 weeks of age. Parallel injections of toxins into wild-type C57/BL6J mice were used as controls.

### RTX-induced Sensory Neuron Denervation

For denervation of TRPV1⁺ cells, resiniferatoxin (Adipogen) or vehicle (0.2% Tween/2% DMSO in normal saline) was administered to 4-week-old mice by IP injection at escalating doses of 30, 70, and 100 μg/kg on 3 consecutive days. Mice were used for experiments at 8 weeks of age.

C. difficile Strains and Spore Preparation

C. difficile 630 Δerm and 630 Δerm (A−/B−) strains were kindly provided by Nigel Minton (University of Nottingham, Nottingham, United Kingdom). C. difficile C. difficile R20291 and M7404 (A−/B+) were kindly provided by Dena Lyras (Monash University, Melbourne, Australia). Cultures were grown in Sporulation broth medium (400 ml) for 5 days. Cells were centrifuged and washed twice in sterile PBS (20 ml), before being shocked with 50% v/v ethanol at room temperature for 1 h. The suspension was then washed five times in PBS, before collecting the spores. For 630 Δerm strains, cultures of C difficile were grown in Brain heart infusion (BD, Difco) overnight before spreading on 70:30 plates. Bacteria were cultivated for 10 days, collected by cotton swab into PBS and then ethanol shocked. Spores were enumerated on ChromID plates and stored at −80.

C. difficile CFUs

Faeces were collected as raw stool from mice one day and two days post gavaging C. difficile spores. Faeces were immediately weighed and resuspended to 50 mg/mL in pre-reduced PBS (allowed to reduce under anaerobic conditions for 24 hours). Live bacteria were enumerated by plating multiple dilutions on pre-reduced commercial ChromID C. difficile plates (Biomerieux, #43871) after incubation for 24 h (37°° C.) in an anerobic chamber (Coy Labs). For experiments involving R20291, cecal content rather than faeces was used for CFU enumeration as it was difficult to collect enough faeces from infected mice. CFUs were blindly counted. 16S rDNA phylotyping

Faecal samples were collected and stored at −80° C. Genomic DNA for downstream 16S rDNA amplicon next generational sequencing was isolated using the ZymoBIOMICSTM-96DNA Kit (Zymo Research, D4309). The 16S amplicon library was prepared in a 96-well format using dual-index barcodes as previously described. Each library was cleaned with the DNA Clean and Concentrator TM-5 Kit (Zymo Research, D4014) and then quantified by qPCR (NEBNext Library Quant Kit, NEB, E7630). 20 pM of DNA were loaded onto an Illumina MiSeq and sequenced (v3, 600-cycle). Primers used for amplification are: Uni16S_V3: TCGTCGGCAGCGTCAGATGTGTATAAGAGACAG-CCTACGGGNGGCWGCAG (1st PCR primer for universal 16S V3V4; “-” stands for 4˜11nt spacer sequences); Uni16S-V4R: GTCTCGTGGGCTCGGAGATGTGTATAAGAGACAG-GGACTACNVGGGTWTCTAAT (1st PCR primer for universal 16S V4); Nextera_i5: AATGATACGGCGACCACCGAGATCTACAC-[i5]-TCGTCGGCAGCGTC (2nd PCR primers, shared between different rDNA amplicons; i5/i7 refers to Nextera indexes); Nextera_i7: CAAGCAGAAGACGGCATACGAGAT-[i7]-GTCTCGTGGGCTCGG.

To generate the Operational Taxonomic Unit (OTU) table for downstream analyses of gut microbiome composition and diversity, Illumina raw reads were de-multiplexed, paired end joined (PEAR v0.9.6), adapter trimmed (Cutadapt v3.1), quality filtered, dereplicated, and denoised (vsearch v2.23.0) and mapped into an OTU table (FAST v1.2.2) Sequences were mapped against the publicly available 16S rDNA databases SILVA (v132) using QIME2 (2022.11) and clustered into OTUs>=97% nucleotide sequence identity. OTU-based microbial community diversity was estimated by calculating the Fisher's alpha diversity index in phyloseq.

C. difficile Growth In vitro

Frozen stock cultures of bacteria were stored at −80° C. in cryogenic vials. C. difficile isolates were grown on brain-heart-infusion supplemented with hemin (50 mg/L) and vitamin K1 (0.25 mg/L) (BHIS) agar plates or in basal media (BSG; proteose peptone (20 g/L), yeast (5 g/L), NaCl (5 g/L), glucose (5 g/L), potassium phosphate dibasic (5 g/L), cysteine (0.5 g/L), hemin (50 mg/L), and vitamin K1 (0.25 mg/L). All isolates were recovered on and grown in pre-reduced media in a Coy anaerobic chamber (Coy labs). To determine the fitness effects of olcegepant or aprepitant on C. difficile, C. difficile 630 Δerm was recovered on a BHIS agar plate and incubated anaerobically at 37° C. for 48 h. A starter culture (1 mL) of BSG was inoculated with C. difficile and grown for 15 h. This starter culture was then used to inoculate a BSG sub-culture (1 mL) at a final dilution of 1:10. This sub-culture was grown for 3 h to an OD600˜0.5 before being used to inoculate experimental cultures at a final dilution of 1:50. The experimental media comprised a modified YCFA media (mYCFA; casitone (2.5 g/L), yeast extract (0.625 g/L), cysteine (0.5 g/L), magnesium sulfate heptahydrate (f.c. 365 μM), calcium chloride dihydrate (f.c. 610 μM), sodium bicarbonate (4 g/L), dipotassium phosphate (0.45 g/L), monopotassium phosphate (0.45 g/L), sodium chloride (0.9 g/L), sodium acetate (2.71 g/L), Hemin (50 mg/L), Vitamin K1 (0.25 mg/L), ferrous sulfate (4 μg/mL), ATCC vitamin mix (1% v/v) supplemented with 0.25% glucose and 20 μM olcegepant, 20 μM aprepitant, or vehicle (0.04% DMSO). Bacterial growth was monitored by measuring the optical density at 600 nm (OD600) of each culture in a 384 well plate with an Epoch2 spectrophotometer (BioTek Instruments) for 24 h.

### Statistical Analysis and Reproducibility

Data were plotted and all statistical analysis was performed using Graphpad Prism. No statistical methods were used to pre-determine sample sizes, rather the experience of the investigators and pilot data determined subsequent sample sizes. Statistical tests and post hoc tests are reported in the Brief Descriptions. The threshold for statistical significance is p <0.05,exact p values are reported for the experiments in each figure and the accompanying source data. For comparisons of multiple inhibitors, comparisons were made to the vehicle group using Dunnett's multiple comparison test. For multiple comparisons between several groups, a Tukcy's post hoc test was utilized. Where data showed a non-parametric distribution, a Kruskal-Wallis test was used followed by post-hoc's test. For tests involving two groups, a Student's Two-tailed T-test was used and for categorical data, Fisher's exact test was used.

For qualitative microscopy experiments, a minimum of N=3 independent animals were utilized per antibody or genetic reporter and the core observations were independently assessed by 3 independent investigators (MR, MM, JM or AS). For qualitative western blots, these were repeated twice, plus an independent antibody was assessed for Calcrl. For in vitro bacterial growth experiments, these were performed twice with 3 wells per condition.

### Example 2. Clinical Trials Targeting Neurogenic Inflammation in C. difficile Infection

A series of clinical trials are performed to demonstrate the ability of antagonists of neurogenic inflammation to treat C. difficile infections in human patients.

First, a trial is performed in which the tested subjects are hospital outpatients diagnosed with C. difficile infection by fecal testing. An initial focus is on aprepitant and the substance P pathway as this medication is well known to be safe in immunocompromised patients. Anti-CGRP agents are an alternative approach.

Treatment groups are as follows: (1) Vancomycin treatment alone (standard of care); (2) Vancomycin treatment and a multi-day course of aprepitant (e.g., three-day course); (3) patients having recurrent infections treated with vancomycin; and (4) patients having recurrent infections treated with vancomycin and a multi-day course of aprepitant (e.g., three-day course).

The primary outcome is time to resolution of infection (e.g., based on symptoms, fecal toxin levels, etc). Time to resolution of infection can be assessed based on nausea, number of unformed bowel movements; stool frequency/character; and quality of life (at baseline, 3-5d, 10d and 21d). A secondary outcome can be incidence of recurrence following infection

Second, a trial is performed in which the tested subjects are hospitalized patients with C. difficile infection. Populations can be adult or pediatric ICU patients with C. difficile infection; or oncology/BMT patients who develop C. difficile infection. Patients can be treated with anti-NK1R (aprepitant) or anti-CGRP agents (Rimegepant, fremanezumab, etc) along with antibiotics (standard of care).

For ICU patients, the primary outcomes could be time to deescalate level of care (e.g., ICU to hospital floor) and/or need for transfusions. For non-ICU patients, the primary outcomes could be time to resolution of infection (e.g., based on symptoms, fecal toxin levels, etc).

Third, a trial is performed in which the tested subjects are patients with frequent episodes of recurrent C. difficile infection. Patients can be treated with long-acting anti-CGRP monoclonal antibodies (e.g., fremanezumab) as a prophylactic agent or following infection.

Fourth, a trial is performed in which the tested subjects are immunocompromised patients or those at high risk of colitis, such as those with underlying IBD. Patients can be treated with an anti-CGRP or anti-NK1R agent concurrent with standard of care (e.g., vancomycin).

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of treating a Clostridioides difficile (C. difficile) infection in a subject, the method comprising administering a therapeutically effective amount of an antagonist of neurogenic inflammation to the subject.

2. The method of claim 1, wherein the antagonist of neurogenic inflammation is an antagonist of a neuropeptide or neuropeptide signaling.

3. The method of claim 2, wherein the neuropeptide is substance P or calcitonin gene-related peptide (CGRP).

4. The method of claim 3, wherein the antagonist of substance P is a molecular agent that targets a substance P peptide or a neurokinin-1 receptor (NK1R) protein, optionally wherein the molecular agent is a small molecule or an antibody.

5. The method of claim 4, wherein the antagonist of substance P is aprepitant, fosaprepitant, netupitant, palonosetron, rolapitant, fosnetupitant, netupitant, or an aprepitant emulsion.

6. The method of claim 3, wherein the antagonist of substance P is a molecular agent that targets a gene encoding a substance P or a neurokinin-1 receptor (NK1R), optionally wherein the molecular agent is an inhibitory nucleic acid.

7. The method of claim 3, wherein the antagonist of CGRP is a molecular agent that targets a CGRP or a CGRP receptor, optionally wherein the molecular agent is a small molecule or an antibody.

8. The method of claim 7, wherein the antagonist of CGRP is fremanezumab, olcegepant, rimegepant, ubrogepant, erenumab, epitinezumab, galcanenzumab, atogepant, telcagepant, UB-13 or zavegepant.

9. The method of claim 3, wherein the antagonist of CGRP is a molecular agent that targets a gene encoding a CGRP or a CGRP receptor, optionally wherein the molecular agent is an inhibitory nucleic acid.

10. The method of claim 3, wherein the CGRP is CGRPα or CGRPβ.

11. The method of any preceding claim, wherein the subject has a C. difficile infection or is suspected of having a C. difficile infection.

12. The method of claim 11, wherein the subject has been diagnosed with a C. difficile infection by fecal testing.

13. The method of claim 11 or 12, wherein the subject has recurrent C. difficile infection.

14. The method of any one of claims 11-13, wherein the subject is a hospitalized patient.

15. The method of any one of claims 1-10, wherein the subject is at risk of contracting a C. difficile infection.

16. The method of claim 15, wherein the subject is an immunocompromised patient or at high risk of colitis, optionally wherein the subject has inflammatory bowel disease, optionally wherein the subject is being treated with a broad-spectrum antibiotic.

17. The method of any preceding claim, wherein administering the antagonist of neurogenic inflammation to the subject results in a decreased period of time to resolution of the infection, relative to a control.

18. The method of any preceding claim, wherein administering the antagonist of neurogenic inflammation to the subject results in a decreased incidence of recurrence following resolution of the infection, relative to a control.

19. The method of any preceding claim further comprising administering a standard of care treatment to the subject.

20. The method of claim 19. wherein the standard of care treatment is an antibiotic treatment, optionally a vancomycin. fidaxomicin, and/or metronidazole treatment.
